(19) United States
(12) Patent Application Publication
(10) Pub. No.: US 2026/0345789 A1
(43) Pub. Date: Nov. 5, 2026

(54) COMPOSITIONS AND METHODS FOR TARGETING TUMOR-ASSOCIATED MACROPHAGES

(71) Applicant: Resolute Science, Inc., San Diego, CA (US)

(72) Inventor: Faith Hemenway BARNETT, San Diego, CA (US)

(21) Appl. No.: 19/157,572

(22) PCT Filed: Feb. 21, 2024
(86) PCT No.: PCT/US2024/016745

§ 371 Date: Aug. 18, 2025

Related U.S. Application Data
(60) Provisional application No. 63/447,626, filed on Feb. 22, 2023.

(51) Int. Cl. A61K 47/54; A61K 31/5517; A61K 45/06; A61K 47/65; A61P 35/00; C07H 7/06

(52) U.S. Cl. CPC A61K 47/549; A61K 31/5517; A61K 45/06; A61K 47/65; A61P 35/00; C07H 7/06

(57) ABSTRACT

The present disclosure relates to compositions that target tumor associated macrophages. The compositions disclosed here preferably comprise a glucan backbone, a tumor associated macrophage targeting moiety, a targeting moiety linker, a payload and optionally a payload linker. The present disclosure also provides methods of making such compositions. The present disclosure also provides methods of treatment using such compositions.

### CROSS-REFERENCE TO RELATED APPLICATION

This application claims priority from U.S. provisional application No. 63/447,626, filed Feb. 22, 2023, entitled “COMPOSITIONS AND METHODS FOR TARGETING TUMOR-ASSOCIATED MACROPHAGES,” the contents of which are incorporated by reference in their entirety.

### BACKGROUND

Soft tissue sarcomas (STS) are rare but deadly cancers of children and adults. The American Cancer Society estimates about 13,000 new STS cases per year in the United States with about 5,130 expected deaths and a 5-year survival for metastatic disease of only 16%. Conventional treatments, including surgery, chemotherapy, and radiotherapy, have yielded limited treatment success for STS. For example, undifferentiated pleomorphic sarcoma (UPS), a highly aggressive adult sarcoma, has a median overall survival for metastatic UPS of only 15.5 months. An adolescent associated aggressive sarcoma, Ewing's sarcoma/primitive neuroectodermal tumor (PNET), has a five-year survival rate of 15% for metastatic cases. Treatment of solid tumors has recently improved with the advent of a broad array of targeted therapies, such as small molecules and biologics. Small molecules have excellent penetrance into solid tumors, yet many of them work by inhibiting signal transduction pathways leading to growth inhibition rather than direct tumor cytotoxicity. If the entire tumor is not killed, cancer cells can develop resistance to the small molecule. Biologics, such as antibody and antibody-drug conjugates (ADC) can have profound potency for certain malignancies and have favorable stability in blood. However, antibodies have limited penetrance into solid tumors, and tumor cells can develop resistance to ADCs through a variety of cellular modifications (Collins et al., “Acquired Resistance to Antibody-Drug Conjugates” Cancers (Basel), 2019, 11(3):394). Thus, new approaches are needed to overcome the limitations of the current state of the art and to further improve the treatment outcomes of STS. In addition, STS encompass over 50 different histologic and molecular subtypes, with each displaying variable clinical behavior (Katz et al., “More Than 50 Subtypes of Soft Tissue Sarcoma: Paving the Path for Histology-Driven Treatments” Am Soc Clin Oncol Educ Book, 2018, 38, 925-938). Due partly to this variability, current STS treatment options have yielded limited efficacy and there is no single or combination treatments that can consistently and effectively treat all STS subtypes.

CD206⁺ cells, particularly macrophages, have been targeted by various molecules in the hopes of delivering diagnostic and therapeutic to sites where such cells assemble. One example of such molecules is found in US 2017/0209584, entitled, “Compositions for Targeting Macrophages and Other CD206 High Expressing Cells and Methods of Treating and Diagnosis.” While the molecules disclosed in this reference and others may target the CD206⁺ cells of interest, the molecules suffer from a number of short comings.

### BRIEF SUMMARY

In one aspect, provided is a composition comprising i) a tumor-associated macrophage-targeting moiety; ii) a glucan backbone, wherein the glucan backbone comprises a plurality of backbone monomers; iii) an active component comprising a pyrrolobenzodiazepine (PBD) dimer, wherein the active component is coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety.

Also provided is a method of delivering an agent to a tumor-associated macrophage comprising contacting the macrophage with a composition described herein.

Also provided is a method of treating cancer, a granulomatous disease, or a nonmalignant tumor in a subject in need thereof, comprising administering to the subject a therapeutically effective amount of a composition described herein.

### BRIEF DESCRIPTION OF THE DRAWINGS

FIG. 1 is a morphology image of mouse M1 cells.

FIG. 2 is a morphology image of mouse M2 cells.

FIG. 3 is a graph showing mouse M1, M2, and 3T3 cell viability results after treatment with the compound of Formula (A-1).

FIG. 4 is a graph showing mouse M1, M2, and 3T3 cell viability results after treatment with the compound of Formula (A-1)

FIG. 5 is a graph showing mouse M1, M2, and 3T3 cell viability results after treatment with the compound of Formula (A-2).

FIG. 6 is a graph showing mouse M1, M2, and 3T3 cell viability results after treatment with the compound of Formula (A-2).

FIGS. 7A and 7B are graphs showing the decrease of weight of mice that were administered increasing concentrations of Formula (A-1).

FIG. 8 is a graph showing the food consumption of the mice administered increasing concentrations of Formula (A-1).

FIGS. 9A-9L are graphs depicting blood hematology parameters between the groups of mice administered increasing concentrations of Formula (A-1).

FIGS. 10A-10T are graphs depicting differences in serum components between the groups of mice administered increasing concentrations of Formula (A-1).

FIG. 11 depicts the decreased tumor burden of mice administered formula (A-1) in a PDX myxofibrosarcoma model.

### DETAILED DESCRIPTION

The present disclosure relates to compositions that target tumor-associated macrophages. The compositions disclosed herein comprise a glucan backbone, a tumor-associated macrophage-targeting moiety, a targeting moiety linker, a payload, and optionally a payload linker. The present disclosure also provides methods of making and using such compositions.

### Chemical Definitions

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkylene” as used herein refers to the same residues as alkyl, but having bivalency. Particular alkylene groups are those having 1 to 20 carbon atoms (a “C₁-C₂₀ alkylene”), having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbamate” refers to the group —O—C(═O)—NH—. Unless specified otherwise, it is understood that the nitrogen atom of the carbamate group is unsubstituted (i.e., bears a hydrogen atom).

“Oxo” refers to the moiety ═O.

Unless otherwise indicated, “aryl,” by itself or as part of another term, means a substituted or unsubstituted monovalent carbocyclic aromatic hydrocarbon radical of the stated number of carbon atoms, typically 6-20 carbon atoms, derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated, an “arylene,” by itself or as part of another term, is an aryl group as defined above which has two covalent bonds (i.e., it is divalent) and can be in the ortho, meta, or para orientations.

Unless otherwise indicated, a “heterocyclyl” by itself or as part of another term, refers to a monovalent substituted or unsubstituted aromatic or non-aromatic monocyclic or bicyclic ring system having from 3 to 9 carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic.

Unless otherwise noted, the heterocyclyl is attached to its pendant group at any heteroatom or carbon atom that results in a stable structure. As such a heteroaryl may be bonded through an aromatic carbon of its aromatic ring system, referred to as a C-linked heteroaryl, or through a non-double-bonded N atom (i.e., not ═N—) in its aromatic ring system, which is referred to as an N-linked heteroaryl. Thus, nitrogen-containing heterocyclyls may be C-linked or N-linked and include pyrrole moieties, such as pyrrol-1-yl (N-linked) and pyrrol-3-yl (C-linked), and imidazole moieties such as imidazol-1-yl and imidazol-3-yl (both N-linked), and imidazol-2-yl, imidazol-4-yl and imidazol-5-yl moieties (all of which are C-linked).

When explicitly given, the size of the ring system of a heterocyclyl or heteroaryl is indicated by the total number of atoms in the ring. For example, designation as a 5- or 6-membered heteroaryl indicates the total number or aromatic atoms (i.e., 5 or 6) in the heteroaromatic ring system of the heteroaryl but does not imply the number of aromatic heteroatoms or aromatic carbons in that ring system. Fused heteroaryls are explicitly stated or implied by context as such and are typically indicated by the number of aromatic atoms in each aromatic ring that are fused together to make up the fused heteroaromatic ring system. For example, a 5,6-membered heteroaryl is an aromatic 5-membered ring fused to an aromatic 6-membered ring in which one or both rings have aromatic heteroatom(s) or where a heteroatom is shared between the two rings.

“Optionally substituted” unless otherwise specified means that a group may be unsubstituted or substituted by one or more (e.g., 1, 2, 3, 4, 5, 6, 7, 8, 9, 10, 11, or 12) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

### Compositions

The compositions disclosed here comprise various components, including a glucan backbone, a tumor-associated macrophage-targeting moiety, a targeting moiety linker, a payload (e.g., pyrrolobenzodiazepine), and optionally a payload linker. The arrangement of these components provides a composition that targets tumor-associated macrophages (TAMs). The disclosed compositions may use the TAMs as receiver cells to pick up, process, and deliver payloads to the tumor environment. For example, sarcomas are characterized by abundant tumor-associated macrophages (TAMs) (Fujiwara et al., 2021). Anti-CD206 immunohistochemisty on human sarcoma microarray, including 59 specimens encompassing 19 sarcoma subtypes, have been shown to have high and relatively uniform expression of this TAM receptor on essentially all specimens. The disclosed compositions may be internalized by cells (e.g., CD206+) present in tumor-associated macrophages. The ability to be internalized by cells present in tumor-associated macrophages allows for the disclosed compositions to deliver payloads (e.g., pyrrolobenzodiazepine) to disease sites where such cells assemble. The present application describes improved compositions and methods for treating cancers, including delivering an agent (e.g., pyrrolobenzodiazepine) to a tumor-associated macrophage by contacting the tumor-associated macrophage with the disclosed composition. The compositions disclosed herein are larger than typical small molecules but smaller than antibody drug conjugates, allowing excellent penetration into targeted locations and minimal leakage to normal tissues, thereby limiting potential toxicities. In addition, while rapidly dividing, mutating and heterogenous cancer cells often develop resistance to traditional therapies, terminally-differentiated macrophages are under no selective pressures to develop resistance to the treatment methods disclosed herein.

The disclosed composition may also target specific receptors, such as CD205 (DEC205), CD206, CD207 (langerin), CD209 (DC-SIGN), CD280 (ENDO180), and CD301 (MGL), which are present on a tumor or near a tumor, allowing targeted delivery of payloads to the tumor.

### Glucan Backbone

The compounds described here comprise a glucan backbone, which is a linear, branched, or circular oligosaccharide or polysaccharide comprising a plurality of glucose monomers. In some embodiments, the plurality of glucose monomers are linked predominantly by C-1→C-6 glycosidic bonds. Other linkages such as α-1,3 or α-1,4 linkages may also be present. In some embodiments, the plurality of glucose monomers are linked by α-1,6 and α-1,3 glycosidic bonds. In some embodiments, the plurality of glucose monomers are linked by a mixture of α-1,6 and α-1,4 glycosidic bonds. A glucan backbone may also be defined as a polymer of glucose wherein the position of glycosidic bonds is varied. A glucan backbone may comprise the alpha or the beta isomer of glucose or a mixture of alpha and beta isomers. Examples of glucan backbones include dextran, a linear or branched compound, and cyclodextrin, a circular glucan. It is to be understood that, in some embodiments, monomers labeled with, for example, a, b, or c, are interspersed within the construct. It is also to be understood that, in some embodiments, the constructs described herein can be block or interspersed, e.g., random.

A glucan backbone may vary in mass and molecular weight, as determined in part by the number of glucose monomers. In some embodiments, a glucan backbone may range in molecular weight from 1-30 kilodaltons (kDa). Preferred embodiments include glucan backbones of approximately 1 kDa, 3 kDa, 6 kDa, 10 kDa, 20 kDa, or 30 kDa. In some embodiments, the glucan backbone may range in molecular mass from 1,000 to 30,000 grams per mole (g/mol). In some embodiments, the glucan backbone may contain glucose monomers ranging from 5 to 167 in number. The glucan backbone can be linear, branched, circular, or combinations thereof. For example, dextran is an example of a linear or branched glucan backbone. Cyclodextrin is another example of a glucan backbone. The backbones described here can be substituted or unsubstituted. For example, a substituted cyclodextrin is a cyclodextrin derivative that is hydrophobic, hydrophilic, ionized, non-ionized, or any other variation thereof.

In some embodiments, the glucan backbone comprises a plurality of backbone monomers, wherein the plurality of backbone monomers comprises a plurality of D-glucose monomers in a α-1,6 glycosidic linkage or beta-1,4 glycosidic linkage. In some embodiments, the plurality of D-glucose monomers is n, wherein n=16 to 111. In some embodiments, n=50 to 65. In some embodiments, the glucan backbone is a linear dextran molecule. In some embodiments, the glucan backbone is a cyclodextrin molecule, comprising 6 to 16 D-glucose monomers.

### Targeting Moiety

The compositions disclosed herein comprise a targeting moiety (e.g., a tumor-associated macrophage-targeting moiety) coupled to a glucan backbone. In some embodiments, the targeting moiety binds to a receptor, including but not limited to, CD205 (DEC205), CD206, CD207 (langerin), CD209 (DC-SIGN), CD280 (ENDO180), or CD301 (MGL). In some embodiments, the targeting moiety is a CD205 targeting moiety. In some embodiments, the targeting moiety is a CD206 targeting moiety. In some embodiments, the targeting moiety is a CD207 targeting moiety. In some embodiments, the targeting moiety is a CD209 targeting moiety. In some embodiments, the targeting moiety is a CD280 targeting moiety. In some embodiments, the targeting moiety is a CD301 targeting moiety. In some embodiments, the target receptor is on a tumor-associated macrophage. In some embodiments, the target receptor is on a cancer or tumor cell. In some embodiments, the targeting moiety comprises mannose, galactose, collagen, fucose, sulfated N-acetylgalactosamine, N-acetylglucosamine, luteinizing hormone, thyroid stimulating hormone, phospholipase A2 or fragments thereof, or a chondroitin sulfate. In some embodiments, the targeting moiety comprises a mannose, including D- and L-isomers thereof. In some embodiments, the targeting moiety comprises a furanose. In some embodiments, the targeting moiety comprises a pyranose. In some embodiments, the targeting moiety is D-mannose. It is understood that the tumor-associated macrophage-targeting moiety may be referred to as the targeting moiety.

In some embodiments, the targeting moieties are attached to between about 10% and about 50% of the glucose residues of the glucan backbone, or between about 20% and about 45% of the glucose residues, or between about 25% and about 40% of the glucose residues. (It should be noted that with reference to a collection or population of the compositions described herein, the MWs referenced herein, as well as the number and degree of conjugation of receptor substrates, leashes, and therapeutic moieties attached to the dextran backbone refer to average amounts for a given quantity of carrier molecules, since the synthesis techniques will result in some variability.)

### Ratio of Targeting Linker to Backbone

The density of a targeting moiety relative to backbone subunits is presented using a targeting moiety to backbone subunit ratio for linear, branched, or circular polysaccharide backbones. For example, degree of substitution (d.s.) is used to communicate the density of targeting moieties on a glucan backbone. The ratio of a targeting moiety to a glucan backbone refers to the number of targeting moieties that substitute a backbone subunit or subunits. For example, a ratio of 1:7 or 1 to 7 means that there is one targeting moiety for every seven glucose subunits in a glucan backbone. The d.s. describes the average number of substituents or substituted positions per unit base. For example, a d.s. of 0.9 means that one backbone subunit is substituted with an average of 0.9 targeting moieties. In some embodiments, the targeting moiety to backbone subunit ratio is from about 1:5 to about 1:25. In some embodiments, the targeting moiety to backbone subunit ratio is from at least 1 to 50 (e.g., at least 1 to 33, at least 1 to 35, at least 1 to 40, or at least 1 to 45) to about 1:5. In some embodiments, the targeting moiety to backbone subunit ratio is from about 1:6 to about 1:19. In some embodiments, the d.s. is from about 0.1 to about 7. In some embodiments, the d.s. is from about 0.5 to 5. In some embodiments, in conjunction with the embodiments above or below, the targeting moiety comprises a mannose.

### Targeting Linker

A targeting linker is a cleavable or a non-cleavable linker that connects a glucan backbone to a targeting moiety. A cleavable linker is capable of being cleaved by an enzyme (e.g., a protease), a change in temperature, a change in pH, a chemical stimulus, or any combination thereof. The cleavable linker may comprise a protease cleavage site. In some embodiments, the cleavable linker is capable of cleavage by a lysosomal protease or an endosomal protease.

The targeting linker may comprise a carbamate group. In some embodiments, the targeting linker comprises a carbamate group and a chain moiety, wherein the carbamate group is connected to a backbone monomer and the chain moiety connects the carbamate group and the targeting moiety. In some embodiments, the chain moiety of the targeting linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the one or more targeting moieties are attached to the glucan backbone through a linker. The linker may be attached at from about 1 to about 50% of the backbone moieties. In some embodiments, the targeting linker comprises a C₁₋₁₂ alkylene chain and a carbamate group, wherein the carbamate group is connected to the backbone monomer and the C₁₋₁₂ alkylene chain connects the carbamate group and the targeting moiety.

### Active Component

An active component is a molecule or a compound that may be used for therapeutic purposes. An active component is also referred to as a payload. An active component may be or comprise a cytotoxic agent or a cytostatic. In some embodiments, a payload may facilitate targeted delivery of the compositions described herein to the macrophages or cancer cells. In some embodiments, the payload is a hydrophobic payload (e.g., topoisomerase inhibitor I, topoisomerase inhibitor II, or temozolomide). In some embodiments, the payload is a hydrophilic payload. In some embodiments, the payload is a therapeutic agent. In some embodiments, the therapeutic agent is selected from a group including, but not limited to, lomustine, epirubicin, topotecan, irinotecan, pemetrexed, docetaxel, oxaliplatin, altretamine, valrubicin, and sarcin.

In some embodiments, the active component comprises a pyrrolobenzodiazepine (PBD) toxin or a pharmaceutically acceptable salt thereof. In some embodiments, the PBD is a dimer. In some embodiments, the active component comprises a PBD dimer of Formula (I) or a pharmaceutically acceptable salt thereof:

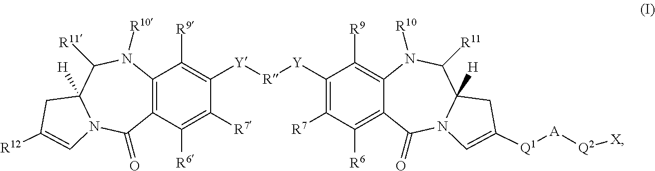

wherein:
A is a C₆-C₁₀ arylene group and X is selected from the group consisting of

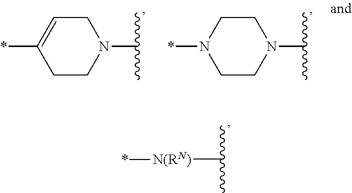

wherein R^{N} is selected from the group consisting of H and C₁-C₄ alkyl; and the asterisk indicates the point of attachment to Q²;
Q¹ and Q² are each independently selected from a single bond, —Z—(CH₂)ₙ—, or —CH═CH—, wherein Z is selected form the group consisting of a single bond, O, S, and NH, and subscript n is from 1 to 3;
R¹² is a C₆-C₁₀ aryl group, optionally substituted by one or more substituents selected form the group consisting of —OR^{12a} and —COOR^{12b}, wherein R^{12a} and R^{12b} are each independently H or C₁-C₆ alkyl;
R⁶, R⁷, and R⁹ are each independently selected from the group consisting of H, R^{b}, —OR^{b}, —SR^{b}, —NR^{b}R^{c}, —NO₂, —SnMe₃, and halo;
   wherein R^{b} and R^{c} are each independently selected from the group consisting of C₁-C₆ alkyl, 4- to 10-membered heterocyclyl, and C₅-C₂₀ aryl, each of which is optionally substituted;
R¹⁰ and R¹¹ are each independently selected from the group consisting of H, —OR^{11a}, and —SO_{Z}M, wherein R^{11a} is H or C₁-C₆ alkyl, subscript Z is 2 or 3, and M is a pharmaceutically acceptable cation; or
R¹⁰ and R¹¹ form a nitrogen-carbon double bond between the nitrogen and carbon atoms to which they are attached;
R″ is a C₃-C₁₂ alkylene, the chain of which is optionally interrupted by one or more heteroatoms selected from the group consisting of O, S, and NR^{N2} wherein R^{N2} is H or C₁-C₄ alkyl, and/or by an C₅-C₁₀ arylene;
Y and Y′ are each independently selected from the group consisting of O, S, and NH; and
R^{6′}, R^{7′}, R^{9′}, R^{10′}, and R^{11′} are independently selected from the same groups as R⁶, R⁷, and R⁹, R¹⁰, and R¹¹, respectively.

In some embodiments, in conjunction with the embodiments above or below, A is a C₆-C₁₀ arylene group and X is

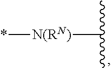

wherein R^{N} is H and the asterisk indicates the point of attachment to Q². In some embodiments, in conjunction with the embodiments above or below, Q¹ is a single bond. In some embodiments, in conjunction with the embodiments above or below, Q² is a single bond. In some embodiments, in conjunction with the embodiments above or below, R¹² is C₆-C₁₀ aryl group, optionally substituted by one or more —OR^{12a}, wherein R^{12a} is H or C₁-C₆ alkyl. In some embodiments, in conjunction with the embodiments above or below, R¹² is phenyl substituted by —OCH₃. In some embodiments, in conjunction with the embodiments above or below, R⁶ is H. In some embodiments, in conjunction with the embodiments above or below, R⁹ is H. In some embodiments, in conjunction with the embodiments above or below, R⁷ is —OR^{b}, wherein R^{b} is C₁-C₆ alkyl. In some embodiments, in conjunction with the embodiments above or below, R⁷ is —OCH₃. In some embodiments, in conjunction with the embodiments above or below, R¹⁰ and R¹¹ form a nitrogen-carbon double bond between the nitrogen and carbon atoms to which they are attached. In some embodiments, in conjunction with the embodiments above or below, R″ is C₃-C₁₂ alkylene. In some embodiments, in conjunction with the embodiments above or below, R″ is a C₃alkylene. In some embodiments, in conjunction with the embodiments above or below, Y is O. In some embodiments, in conjunction with the embodiments above or below, Y′ is O. In some embodiments, in conjunction with the embodiments above or below, R^{6′}, R^{7′}, R^{9′}, R^{10′}, and R^{11′} are selected from the same groups as R⁶, R⁷, and R⁹, R¹⁰, and R¹¹, respectively.

In some embodiments, the active component comprises a PBD dimer of Formula (I-A) or a pharmaceutically acceptable salt thereof:

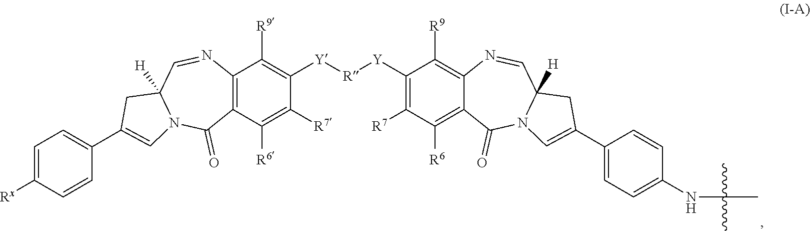

wherein Y, Y′, R″, R⁶, R^{6′}, R⁷, R^{7′}, R⁹, R^{9′}, and R^{x} are as defined in Formula (I).

In some embodiments, the active component comprises a PBD dimer of Formula (I-B) or a pharmaceutically acceptable salt thereof:

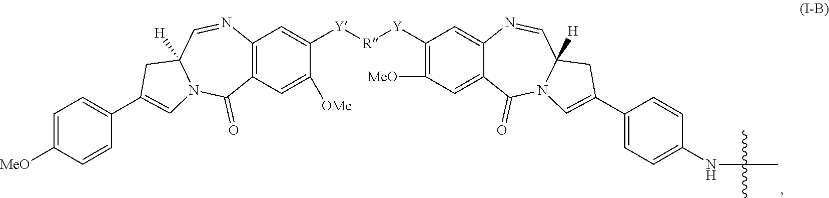

wherein Y, Y′, and R″ are as defined in Formula (I).

In some embodiments, the active component comprises a PBD dimer of Formula (I-C) or a pharmaceutically acceptable salt thereof:

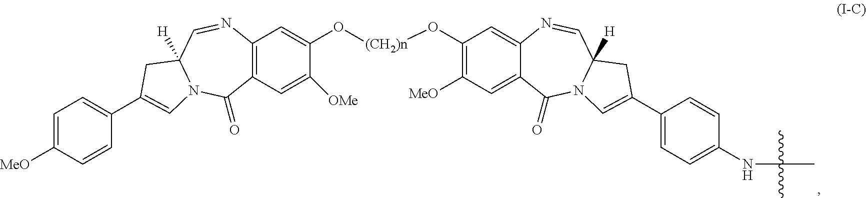

wherein n=3 to 12. In some embodiments, n=3. In some embodiments, n=4. In some embodiments, n=5. In some embodiments, n=6. In some embodiments, n=3. In some embodiments, n=7. In some embodiments, n=8. In some embodiments, n=9. In some embodiments, n=10. In some embodiments, n=11. In some embodiments, n=12.

In some embodiments, the composition comprises a PBD dimer of Formula (I-D) or a pharmaceutically acceptable salt thereof:

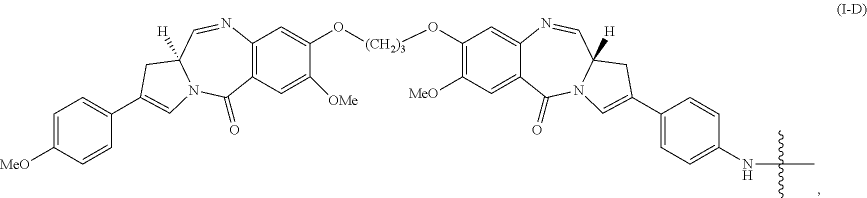

### Payload Linker

In certain embodiments the active component or payload is coupled directly to the glucan backbone. In some embodiments, the active component is connected to a glucan backbone via a linker. The linker can be cleavable or non-cleavable. In some embodiments, the one or more therapeutic agent is attached via a biodegradable linker. In some embodiments, the biodegradable linker is acid sensitive, such as a hydrazone linker. The use of an acid sensitive linker enables the drug to be transported into the cell and allows for the release of the drug substantially inside of the cell. In some embodiments, the payload linker is a Val-Cit linker.

The payload linker may comprise a carbamate group. In some embodiments, the payload linker comprises a carbamate group and a chain moiety, wherein the carbamate group is connected to a backbone monomer and the chain moiety connects the carbamate group and the active component. In some embodiments, the chain moiety of the payload linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the molar ratio between the targeting moiety (e.g., mannose) and the PBD toxin is from about 1:10 to about 10:1. In some embodiments, the molar ratio between the targeting moiety and the PBD toxin is at least about 1:10 (e.g., about 1:9, 1:8, 1:7, 1:6, 1:5, 1:4, 1:3, 1:2, 1:1, 2:1, 3:1, and 4:1). In some embodiment, the molar ratio between the targeting moiety and the PBD toxin is about 1:1. In some embodiment, the molar ratio between the targeting moiety and the PBD toxin is about 1:2. In some embodiment, the molar ratio between the targeting moiety and the PBD toxin is about 1:3. In some embodiment, the molar ratio between the targeting moiety and the PBD toxin is about 1:4.

In some embodiments, the payload linker comprises a —C(O)—C₁₋₁₂ alkylene chain and a carbamate group, wherein the carbamate group is connected to the backbone monomer and the —C(O)—C₁₋₁₂ alkylene chain connects the carbamate group and the payload.

### Secondary Payloads and Linkers

In addition to the payloads, the compounds disclosed here can encompass the inclusion of secondary agents that can be coupled to the glucan backbone to add additional functional capabilities. Typically, the secondary payload is coupled to the linker in any manner similar to what may be used to couple the targeting moiety to the targeting linker.

In some embodiments, a secondary payload may facilitate targeted delivery of the compositions described herein to the macrophages or cancer cells. In some embodiments, the secondary payload is a hydrophobic payload (e.g., topoisomerase inhibitor I, topoisomerase inhibitor II, or temozolomide). In some embodiments, the secondary payload is a hydrophilic payload.

A secondary payload can encompass, for example, additional agents for imaging, therapy, or for other purposes. Specifically, in one embodiment, combinations of therapeutic and imaging agents can be linked to the glucan backbone to combine diagnostic and therapeutic functionalities. In another embodiment, various amino acids, such as cysteine or lysine can be coupled to the linker to crosslink the molecule to a target.

A secondary payload linker is a cleavable or a non-cleavable linker that connects a glucan backbone to a secondary payload moiety. A cleavable linker is capable of being cleaved by an enzyme (e.g., a protease), a change in temperature, a change in pH, a chemical stimulus, or any combination thereof. The cleavable linker may comprise a protease cleavage site. In some embodiments, the cleavable linker is capable of cleavage by a lysosomal protease or an endosomal protease.

The secondary payload linker may comprise a carbamate group. In some embodiments, the secondary payload linker comprises a carbamate group and a chain moiety, wherein the carbamate group is connected to a backbone monomer and the chain moiety connects the carbamate group and the secondary agent. Herein, a carbamate functional group takes the plain and ordinary meaning derived from the field of organic chemistry. In some embodiments, the chain moiety of the secondary payload linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, Cl—C₁₂ alkyl, Cl—C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the one or more secondary payload moieties are attached to the glucan backbone through a linker. The linker may be attached at from about 1 to about 50% of the backbone moieties.

In some embodiments, the composition described herein comprises a compound of Formula (A-1) or a pharmaceutically acceptable salt thereof:

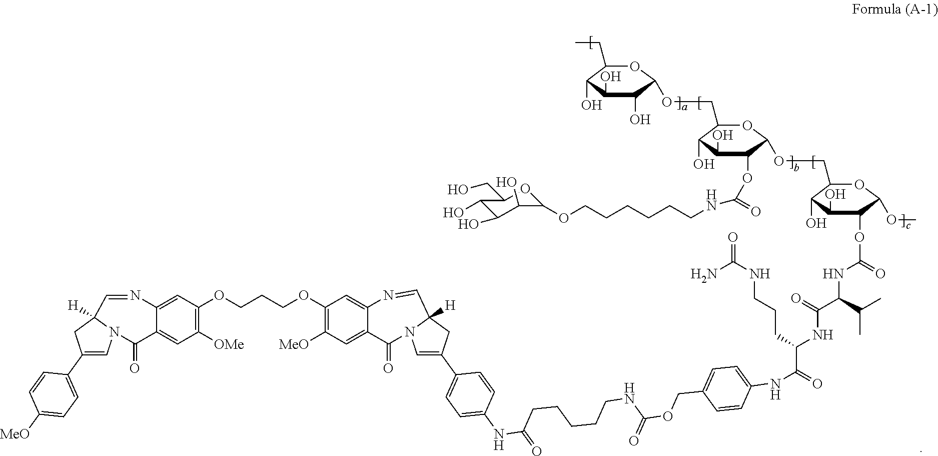

In some embodiments, monomers of the types labelled with a, b, or c in the compound of Formula (A-1) may be in a block co-polymer arrangement or may be interspersed (e.g., randomly arranged) within the polymer or any combination thereof unless otherwise indicated. In some embodiments, a, b, and c of Formula (A-1) may each independently refer to an integer of 0, at least 1, from about 1 to about 165, from about 16 to about 111, from about 50 to about 65, or from about 6 to about 6. In some embodiments, the glucan backbone of Formula (A-1) is linear, branched, circular, or combinations thereof. In some embodiments, an end group of the glucan backbone of Formula (A-1) may be a hydroxy end group of the monomer. In some embodiments, an end group of the glucan backbone of Formula (A-1) may be any end groups recognizable by one skilled in the art.

In some embodiments, the glucan backbone of Formula (A-1) is linear, branched, circular, or combinations thereof; a, b, and c is each independently elected from the group consisting of: an integer of 0, an integer of at least 1, an integer from about 1 to about 165, an integer from about 16 to about 111, an integer from about 50 to about 65, and an integer from about 6 to about 16; and the end group of the glucan backbone is a hydroxy end group of the monomer. In some embodiments, the glucan backbone of Formula (A-1) is circular, and a, b, and c is each independently elected from the group consisting of: an integer of 0, an integer of at least 1, an integer from about 1 to about 165, an integer from about 16 to about 111, an integer from about 50 to about 65, and an integer from about 6 to about 16. In some embodiments, the glucan backbone of Formula (A-1) is linear; a, b, and c is each independently elected from the group consisting of: an integer of 0, an integer of at least 1, an integer from about 1 to about 165, an integer from about 16 to about 111, an integer from about 50 to about 65, and an integer from about 6 to about 16; and the end group of the glucan backbone is a hydroxy end group of the monomer. In some embodiments, the glucan backbone of Formula (A-1) is branched; a, b, and c is each independently elected from the group consisting of: an integer of 0, an integer of at least 1, an integer from about 1 to about 165, an integer from about 16 to about 111, an integer from about 50 to about 65, and an integer from about 6 to about 16; and the end group of the glucan backbone is a hydroxy end group of the monomer.

In some embodiments, in combination with the embodiments above or below, the glucan backbone of Formula (A-1) is about 6 kDa, wherein the glucan backbone is a dextran. In some embodiments, in combination with the embodiments above or below, the a, b, and c groups of Formula (A-1) are interspersed. In some embodiments, in combination with the embodiments above or below, the end groups of the glucan backbone is a natural end group of glucose, such as a hydroxy end group. In some embodiments, in combination with the embodiments above or below, the ratio of the targeting moiety to backbone monomers of Formula (A-1) is about 1:30 to 1:40 (e.g., 1:33) or about 1:33 to 1:40 (e.g., 1:37). In some embodiments, in combination with the embodiments above or below, the ratio of the PBD to mannose of Formula (A-1) is about 1:1 to 1:3 (e.g., 1:1) or about 1:3 to 1:5 (e.g., 1:4).

In some embodiments, provided herein is a compound of Formula (A-1) wherein the glucan backbone is a dextran; the molecular weight of the glucan backbone is about 6 kDa; the a, b, and c groups are interspersed; the end groups of the glucan backbone is a natural end group of glucose, such as a hydroxy end group; the ratio of the targeting moiety to backbone monomers of is about 1:30 to 1:40 (e.g., 1:33); and the ratio of the PBD to mannose is about 1:1 to 1:3 (e.g., 1:1).

In some embodiments, provided herein is a compound of Formula (A-1) wherein the glucan backbone is a dextran; the molecular weight of the glucan backbone is about 6 kDa; the a, c, and d groups are interspersed; the end groups of the glucan backbone is a natural end group of glucose, such as a hydroxy end group; the ratio of the targeting moiety to backbone monomers of is about 1:33 to 1:40 (e.g., 1:37); and the ratio of the PBD to mannose is about 1:3 to 1:5 (e.g., 1:4).

In some embodiments, the composition described herein comprises a compound of Formula (A-2) or a pharmaceutically acceptable salt thereof:

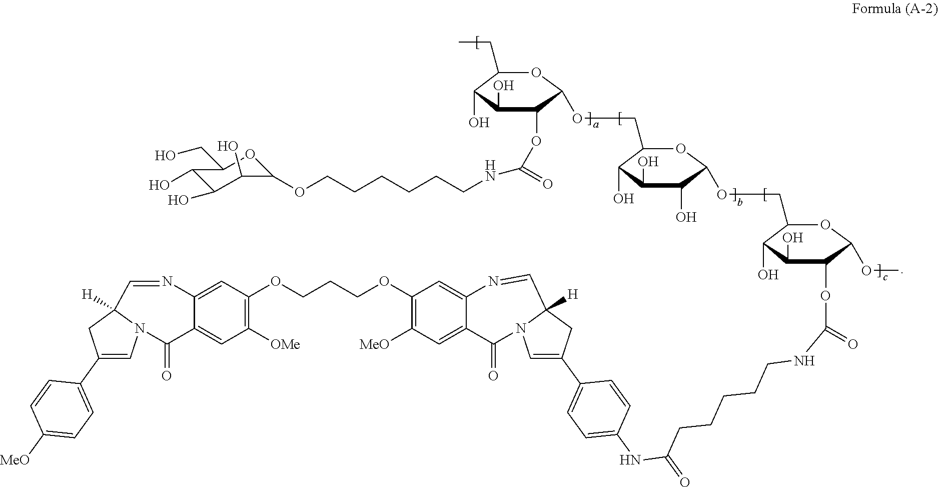

In some embodiments, monomers of the types labelled with a, b, or c in the compound of Formula (A-2) may be in a block co-polymer arrangement or may be interspersed (e.g., randomly arranged) within the polymer or any combination thereof unless otherwise indicated. In some embodiments, a, b, and c of Formula (A-2) may each independently refer to an integer of 0, at least 1, from about 1 to about 165, from about 16 to about 111, from about 50 to about 65. or from about 6 to about 6. In some embodiments, the glucan backbone of Formula (A-2) is linear, branched, circular, or combinations thereof. In some embodiments, an end group of the glucan backbone of Formula (A-2) may be a hydroxy end group of the monomer. In some embodiments, an end group of the glucan backbone of Formula (A-2) may be any end groups recognizable by one skilled in the art.

In some embodiments, the glucan backbone of Formula (A-2) is linear, branched, circular, or combinations thereof; a, b, and c is each independently elected from the group consisting of: an integer of 0, an integer of at least 1, an integer from about 1 to about 165, an integer from about 16 to about 111, an integer from about 50 to about 65, and an integer from about 6 to about 16; and the end group of the glucan backbone is a hydroxy end group of the monomer. In some embodiments, the glucan backbone of Formula (A-2) is circular, and a, b, and c is each independently elected from the group consisting of: an integer of 0, an integer of at least 1, an integer from about 1 to about 165, an integer from about 16 to about 111, an integer from about 50 to about 65, and an integer from about 6 to about 16. In some embodiments, the glucan backbone of Formula (A-2) is linear; a, b, and c is each independently elected from the group consisting of: an integer of 0, an integer of at least 1, an integer from about 1 to about 165, an integer from about 16 to about 111, an integer from about 50 to about 65, and an integer from about 6 to about 16; and the end group of the glucan backbone is a hydroxy end group of the monomer. In some embodiments, the glucan backbone of Formula (A-2) is branched; a, b, and c is each independently elected from the group consisting of: an integer of 0, an integer of at least 1, an integer from about 1 to about 165, an integer from about 16 to about 111, an integer from about 50 to about 65, and an integer from about 6 to about 16; and the end group of the glucan backbone is a hydroxy end group of the monomer.

In some embodiments, in combination with the embodiments above or below, the glucan backbone of Formula (A-2) is about 6 kDa, wherein the glucan backbone is a dextran. In some embodiments, in combination with the embodiments above or below, the a, b, and c groups of Formula (A-2) are interspersed. In some embodiments, in combination with the embodiments above or below, the end groups of the glucan backbone is a natural end group of glucose, such as a hydroxy end group. In some embodiments, in combination with the embodiments above or below, the ratio of the targeting moiety to backbone monomers of Formula (A-2) is about 1:30 to 1:40 (e.g., 1:33) or about 1:33 to 1:40 (e.g., 1:37). In some embodiments, in combination with the embodiments above or below, the ratio of the PBD to mannose of Formula (A-2) is about 1:1 to 1:3 (e.g., 1:1) or about 1:3 to 1:5 (e.g., 1:4).

In some embodiments, provided herein is a compound of Formula (A-2) wherein the glucan backbone is a dextran; the molecular weight of the glucan backbone is about 6 kDa; the a, b, and c groups are interspersed; the end groups of the glucan backbone is a natural end group of glucose, such as a hydroxy end group; the ratio of the targeting moiety to backbone monomers of is about 1:30 to 1:40 (e.g., 1:33); and the ratio of the PBD to mannose is about 1:1 to 1:3 (e.g., 1:1).

In some embodiments, provided herein is a compound of Formula (A-2) wherein the glucan backbone is a dextran; the molecular weight of the glucan backbone is about 6 kDa; the a, c, and d groups are interspersed; the end groups of the glucan backbone is a natural end group of glucose, such as a hydroxy end group; the ratio of the targeting moiety to backbone monomers of is about 1:33 to 1:40 (e.g., 1:37); and the ratio of the PBD to mannose is about 1:3 to 1:5 (e.g., 1:4).

### Therapeutic Methods

The compositions disclosed herein may be used to treat cancer or non-malignant tumors (e.g., meningiomas hemangioblastomas or giant cell tumors) in a subject in need thereof. Treatment comprises administering to the subject a therapeutically effective amount of a composition disclosed herein. The compositions disclosed herein may also be used to treat a granulomatous disease (e.g., sarcoidosis) or chronic inflammatory disorders (e.g., rheumatoid arthritis) in a subject in need thereof. The compositions disclosed herein may also be used to treat a lysosomal storage disease. For example, a lysosomal storage disease includes, but is not limited to, Cholesterly ester storage disease, Wolman disease, Hunter syndrome, Hurler's disease, Fabry disease, Gaucher disease, Krabb disease (globoid cell leukodystrophy) Metachromatic leukodystrophy, Niemann-Pick disease, Sandhoff disease, Tay-Sachs disease, Batten disease, Cystinosis, Danon disease, and Pompe disease. The compositions disclosed herein may also be used to treat macrophage-dependent diseases. In some embodiments, nonmalignant tumors include, but are not limited to, meningioma of all grades (e.g., grade 1 meningioma, grade 2 meningioma, or grade 3 meningioma), schwannomas, schwannomatosis, neurofibromas, neurofibromatosis type 1 (NF1), or neurofibromatosis type 2 (NF2).

In some embodiments, the cancer can be any cell in a subject undergoing unregulated growth. The cancer can be any cancer cell capable of metastasis. For example, the cancer can be a sarcoma, lymphoma, leukemia, carcinoma, blastoma, or germ cell tumor. In some embodiment, the cancer is selected from the group consisting of: carcinoma, lymphoma, blastoma, sarcoma, leukemia, lymphoid malignancies, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric cancer, stomach cancer, gastrointestinal cancer, squamous cell of the esophagus, hepatocellular carcinoma, pancreatic cancer, glioblastoma, cervical cancer, ovarian cancer, liver cancer, bladder cancer, hepatoma, breast cancer (e.g., HR+/HER2− breast cancer, HR−/HER2− breast cancer, HR+/HER2+ breast cancer, HR−/HER2+ breast cancer, triple negative breast cancer, or inflammatory breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, Merkel cell carcinoma, multiple myeloma, acute myeloid leukemia (AML), hemangioblastomas, and schwannomas. In some embodiment, the non-malignant tumor is meningiomas hemangioblastomas or giant cell tumors.

Methods of treating or preventing diseases or disorders are provided using the disclosed compositions. The disclosed compositions can be used for targeting CD205 (DEC205), CD206, CD207 (langerin), CD209 (DC-SIGN), CD280 (ENDO180), or CD301 (MGL), which are present on a tumor or near a tumor. The disclosed compounds can be used for targeting of macrophages for treatment of intracellular pathogens (M. tuberculosis, F. tularensis, S. typhi). The disclosed compounds can be used to target tumor-associated macrophages, e.g. to be used for treating cancer. Macrophage-related and other CD205 (DEC205), CD206, CD207 (langerin), CD209 (DC-SIGN), CD280 (ENDO180), or CD301 (MGL) high expressing cell-related diseases for which the compositions and methods herein may be used include, but are not limited to: acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behcet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barre syndrome (GBS), Hashimoto's encephalopathy, Hashimoto's thyroiditis, heart disease, Henoch-Schonlein purpura, herpes gestationis (aka gestational pemphigoid), hidradenitis suppurativa, histocytosis, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, Rosai-Dorfman disease, sarcoidosis, schizophrenia, Schmidt syndrome, Schnitzler syndrome, scleritis, scleroderma, sepsis, serum Sickness, Sjogren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

One of ordinary skill in the art will appreciate that various kinds of molecules and compounds be delivered to a cell or tissue using the disclosed compounds.

In one aspect, provided herein is a method of treating tuberculosis comprising administering to a subject in need thereof a compound as described herein.

In another aspect, provided herein is a method of treating a macrophage-mediated disorder comprising administering to a subject in need thereof an effective amount of a compound as described herein; and detecting the detection label at a predetermined location in the subject.

In another aspect, provided herein is a method of treating a macrophage-mediated disorder comprising administering to a subject in need thereof an effective amount of a compound as described herein.

In another aspect, provided herein is a method of targeting tumor-associated macrophages comprising administering to a subject in need thereof an effective amount of a compound as described herein.

In another aspect, provided herein is a method according to any of those described herein, wherein a linker is used to attach the one or more targeting moieties and one or more therapeutic agents.

In another aspect, provided herein is a method according to any of those described herein, wherein the disease is rheumatoid arthritis.

In another aspect, provided herein is a method according to any of those described herein, wherein the disorder is cancer.

In another aspect, provided herein is a method according to any of those described herein, wherein the cancer is a sarcoma, lymphoma, leukemia, carcinoma, blastoma, melanoma, or germ cell tumor.

### Combination Therapy

In some embodiments, the compositions disclosed herein may be administered in combination with a second therapeutic agent. In some embodiments, the compositions disclosed herein is co-administered to a patient with a second therapeutic agent, an adjuvant therapy, or radiation therapy. In some embodiments, at least two compounds or compositions are administered to the patient at the same time, such that effective amounts or concentrations of each of the two or more compounds may be found in the patient at a given point in time. Although compositions according to the present disclosure may be co-administered to a patient at the same time, co-administration embraces both administration of two or more agents at the same time or at different times, provided that effective concentrations of all co-administered compounds or compositions are found in the subject at a given time. In certain preferred aspects of the present disclosure, one or more of the present compositions described above, are co-administered in combination with at least one additional bioactive agent.

### Administration

The compositions as described herein may in certain embodiments be administered in single or divided unit doses by the oral, parenteral or topical routes. Administration of the compositions may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, by inhalation spray, rectally, vaginally, or via an implanted reservoir, among other routes of administration. The most effective dosage form will depend upon the pharmacokinetics of the particular agent chosen as well as the severity of disease in the patient.

The disclosed compositions can be administered via any suitable method. The disclosed compositions can be administered parenterally into the parenchyma or into the circulation so that the disclosed compounds reach target tissues (e.g., where cancer cells may be located). The disclosed compositions can be administered directly into or adjacent to a tumor mass. The disclosed compositions can be administered intravenously. In still other embodiments, the disclosed compositions can be administered orally, intraperitoneally, intramuscularly, subcutaneously, intracavity, or transdermally.

Parenteral administration of the compounds, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

### Formulation

In an additional aspect, the description provides therapeutic or pharmaceutical compositions comprising an effective amount of a composition according to the present disclosure, optionally in combination with a pharmaceutically acceptable carrier, additive or excipient.

A typical formulation is prepared by mixing the compositions of the present disclosure with excipients, such as carriers and/or diluents. Suitable carriers, diluents and other excipients are well known to those skilled in the art and include materials such as carbohydrates, waxes, water soluble and/or swellable polymers, hydrophilic or hydrophobic materials, gelatin, oils, solvents, water and the like. The particular carrier, diluent or other excipient used will depend upon the means and purpose for which the compound is being applied. Other pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives.

A patient or subject in need of therapy using compositions according to the present disclosure can be treated by administering to the patient (subject) an effective amount of the composition according to the present disclosure. The composition is included in the pharmaceutically acceptable carrier or diluent in an amount sufficient to deliver to a patient a therapeutically effective amount for the desired indication, without causing serious toxic effects in the patient treated. It should be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

### Kits

In some aspects, kits for use in cancer treatment are provided. Such kits can include a composition described herein.

In some embodiments, the kit can include instructions for use in any of the therapeutic methods described herein. The included instructions can provide a description of administration of the pharmaceutical compositions to a subject to achieve the intended activity, e.g., treatment of a disease or condition such as cancer, in a subject. In some embodiments, the instructions relating to the use of the pharmaceutical compositions described herein can include information as to dosage, dosing schedule, and route of administration for the intended treatment. The containers can be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

In some embodiments, the kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. In some embodiments, a kit can have a sterile access port (for example, the container can be an intravenous solution bag or a vial having a stopper pierceable by a hypodermic injection needle).

In some embodiments, the kits provided herein include an additional therapeutic agent useful in treating a cancer as described herein.

### General Synthetic Methods

Compositions of the present disclosure will now be described by reference to illustrative synthetic schemes for their general preparation below and the specific examples that follow. Artisans will recognize that, to obtain the various compositions herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or side chain groups) from reaction conditions, and that such groups are removed under standard conditions when appropriate.

Chromatography, recrystallization and other conventional separation procedures may also be used with intermediates or final products where it is desired to obtain a particular isomer of a compound or to otherwise purify a product of a reaction.

General methods of preparing compositions described herein are depicted in exemplified methods below.

In some embodiments, the compositions of described herein can be synthesized according to the procedure as shown in Scheme A1.

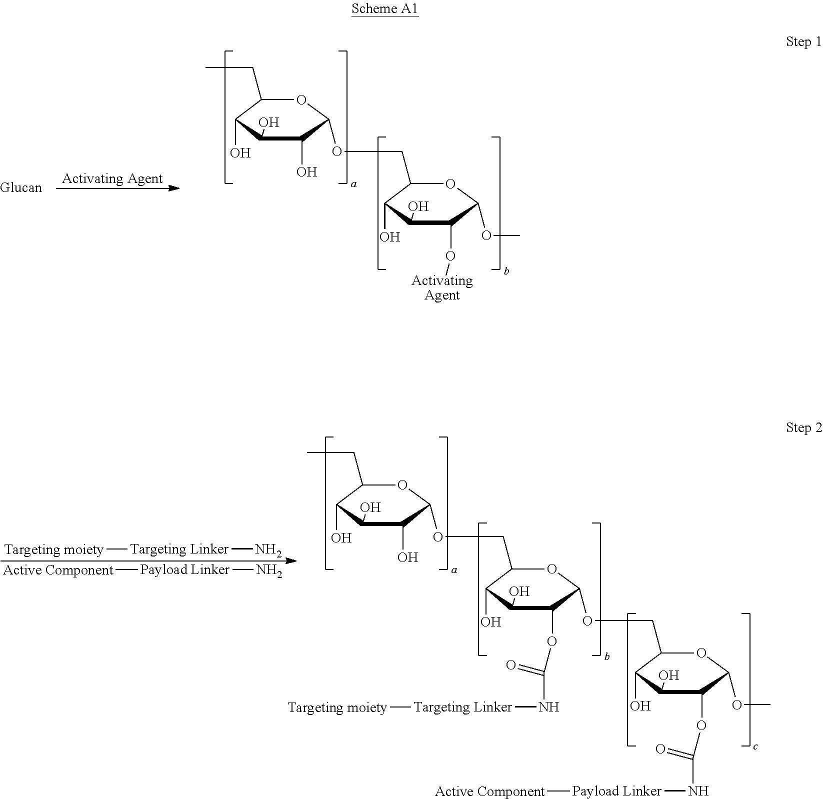

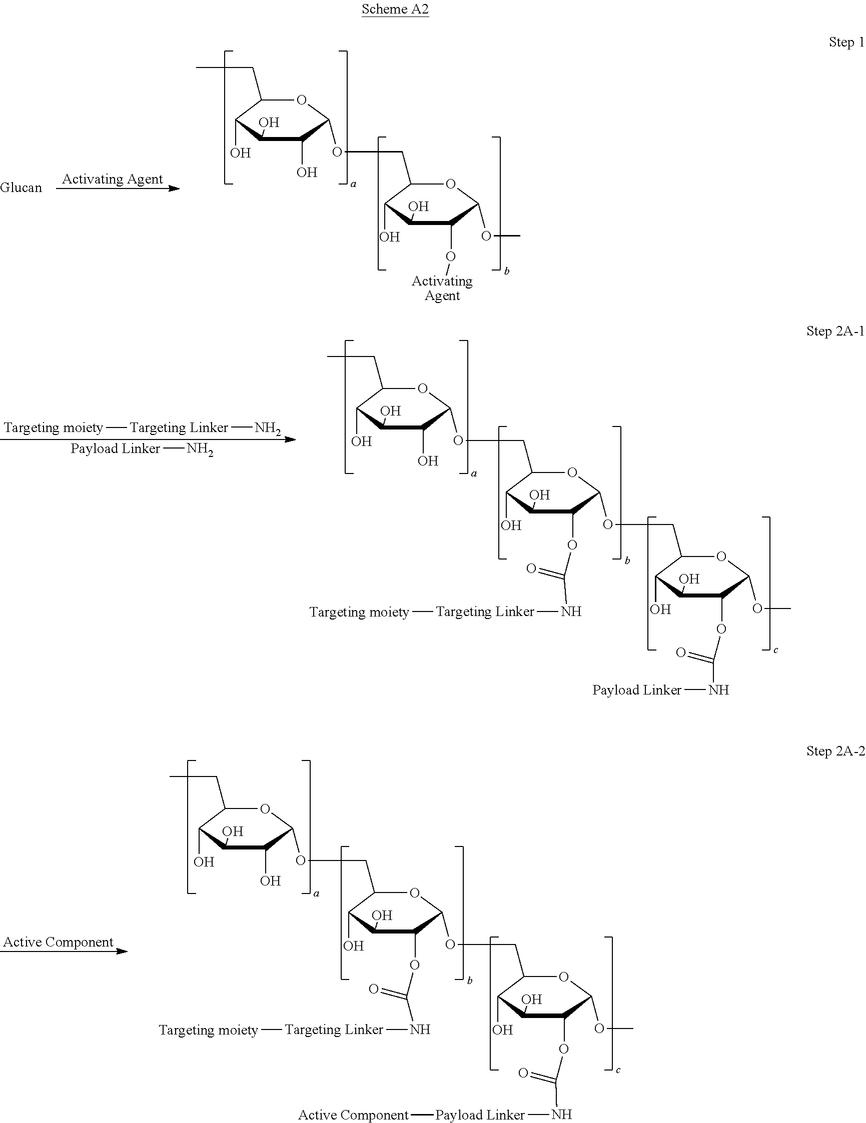

As can be seen in the above schemes, a glucan compound (such as a dextran or a cyclodextrin) is reacted with an activating agent. The resulting activated glucan derivative can then be reacted with the appropriate reagents to introduce a targeting moiety coupled to the glucan backbone via a targeting linker, as well as an active component linked to the glucan backbone via a payload linker. A skilled artisan will recognize that the above schemes are illustrative and that the various reagents and order of synthetic steps can be varied as required for obtaining the intended final products. For example, a, b, and c may each independently refer to an integer of 0, at least 1, at least 1, from about 1 to about 165, from about 16 to about 111, from about 50 to about 65, or from about 6 to about 16. It is to be understood that monomers of the types labelled with a, b, or c may be in a block co-polymer arrangement or may be interspersed (e.g., randomly arranged) within the polymer or any combination thereof unless otherwise indicated. It is also to be understood that the glucan backbone in the above scheme may be linear, branched, circular, or combinations thereof. Groups not specified in the above schemes, such as any end groups to the glucan backbone, may be any end groups recognizable by one skilled in the art. For example, an end group of the glucan backbone may be a hydroxy end group of the monomer.

### EXAMPLES

The following examples are included for illustrative purposes only and are not intended to limit the scope of the disclosure.

### Example 1. Synthesis of PBD Dimer-NH₂(Compound X-1)

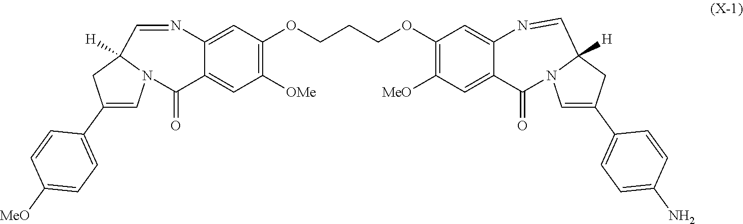 [SMILES: COc1ccc(C2=CN3C(=O)c4cc(OC)c(OCCCOc5cc6c(cc5OC)C(=O)N5C=C(c7ccc(N)cc7)C[C@H]5C=N6)cc4N=C[C@@H]3C2)cc1]

### Model Reaction: Coupling of Aniline to Potential Linker Moiety Fmoc-Val-Cit-PAB-PNP

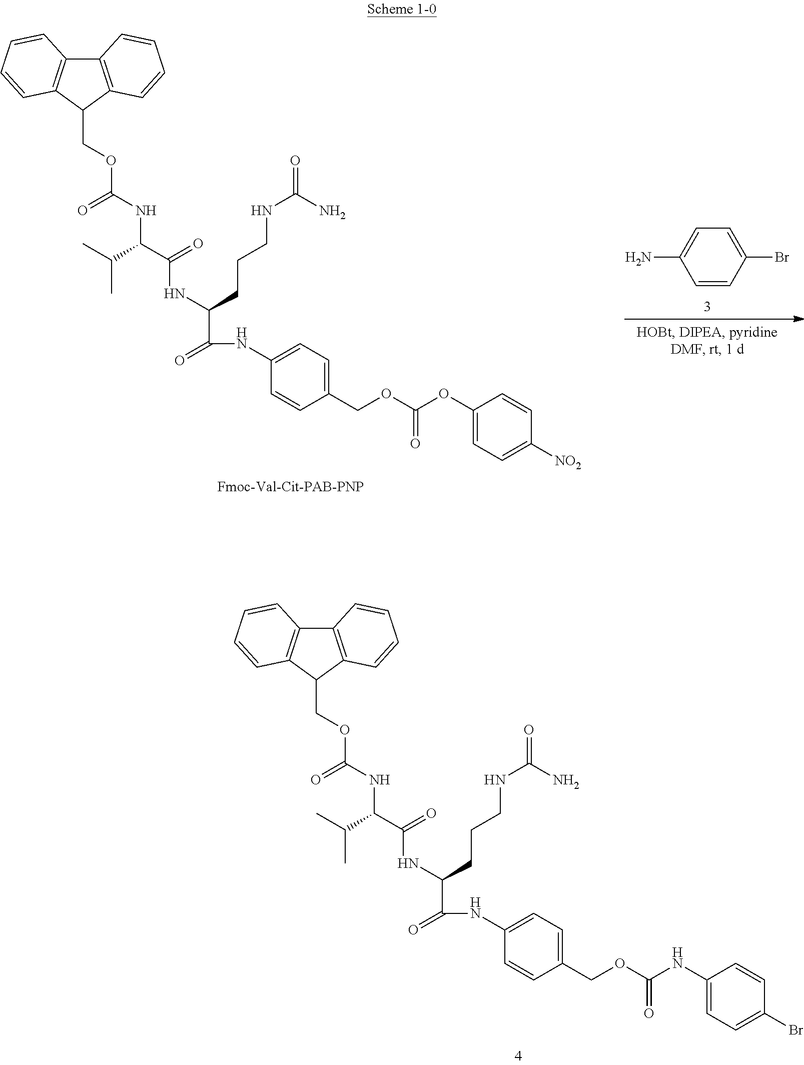 [SMILES: CC(C)[C@H](NC(=O)OCC1c2ccccc2-c2ccccc21)C(=O)N[C@@H](CCCNC(N)=O)C(=O)Nc1ccc(COC(=O)Oc2ccc([N+](=O)[O-])cc2)cc1 ; Nc1ccc(Br)cc1 ; CC(C)[C@H](NC(=O)OCC1c2ccccc2-c2ccccc21)C(=O)N[C@@H](CCCNC(N)=O)C(=O)Nc1ccc(COC(=O)Nc2ccc(Br)cc2)cc1]

To test the feasibility of connecting the PBD dimer to a potential linker Fmoc-Val-Cit-PAB-PNP and form an active component-payload linker moiety, which could serve as a precursor for the compositions described herein, a model reaction was conducted following Scheme 1-0 and compound 4 was successfully synthesized. This clearly demonstrated the feasibility of coupling an aniline unit with a Fmoc-Val-Cit-PAB-PNP linker moiety.

### Step 1. Synthesis of 4-((tert-butyldiphenylsilyl)oxy)-5-methoxy-2-nitrobenzoic acid (Compound A)

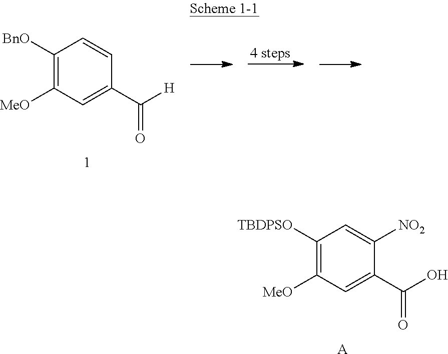

4-((tert-butyldiphenylsilyl)oxy)-5-methoxy-2-nitrobenzoic acid (compound A) was synthesized from compound 1, as shown in Scheme 1-1.

### Step 2. Synthesis of (3R,5S)-5-(((tert-butyldiphenylsilyl)oxy)methyl)pyrrolidin-3-ol (Compound B)

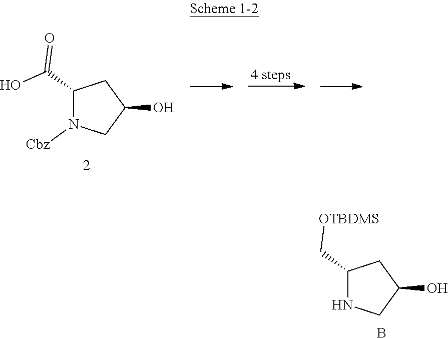

(3R,5S)-5-(((tert-butyldiphenylsilyl)oxy)methyl)pyrrolidin-3-ol (compound B) was synthesized from compound 2, as shown in Scheme 1-2. Particularly, the steps 1, 2, and 3 out of the 4 steps are further shown in Scheme 1-2-1, 1-2-2, and 1-2-3 respectively.

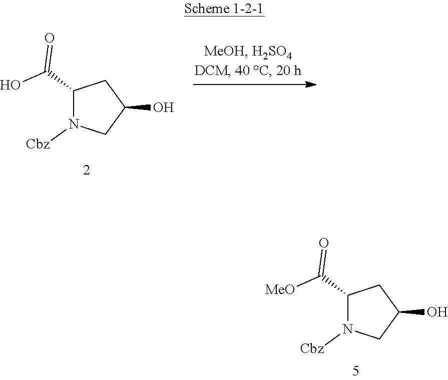 [SMILES: O=C(O)[C@@H]1C[C@@H](O)CN1C(=O)OCc1ccccc1 ; COC(=O)[C@@H]1C[C@@H](O)CN1C(=O)OCc1ccccc1]

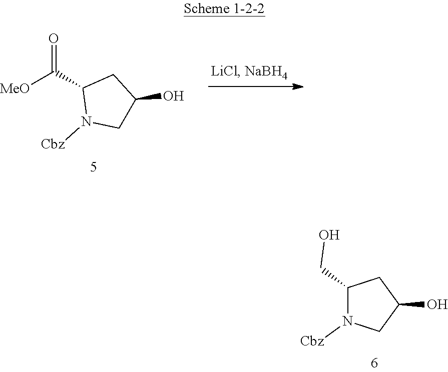 [SMILES: COC(=O)[C@@H]1C[C@@H](O)CN1C(=O)OCc1ccccc1 ; O=C(OCc1ccccc1)N1C[C@H](O)C[C@H]1CO]

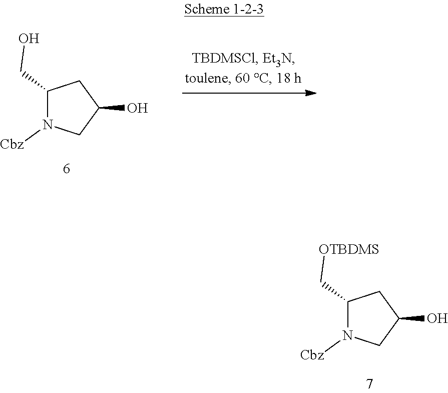 [SMILES: O=C(OCc1ccccc1)N1C[C@H](O)C[C@H]1CO ; CC(C)(C)[Si](C)(C)OC[C@@H]1C[C@@H](O)CN1C(=O)OCc1ccccc1]

In step 1, as shown in Scheme 1-2-1, compound 2 was reacted with MeOH and H₂SO₄ in DCM under 40 °C for 20 hours and afforded compound 5 (99.4 g in 94% yield). In step 2, as shown in Scheme 1-2-2, 5.00 g LiCl (1.3 eq.) and NaBH₄ (1.3 eq.) was mixed in THF/water and stirred at 17 °C for 5 hours. Then additional LiCl (0.6 eq.) and NaBH₄ (0.6 eq.) was added and stirred at room temperature for 19 hours. After 19 hours, TLC showed no starting material, and compound 6 was formed (3.93 g in 87% yield). In step 3, the compound 6 was mixed with TBDMSCl and Et₃N and stirred in toluene under 60 °C for 18 hours. Compound 7 was successfully synthesized.

### Step 3. Synthesis of Building Block (Compound C)

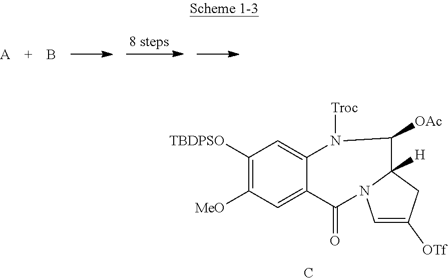

The building block for active component-NH₂ (compound C) was synthesized from compound A and compound B. Particularly, the steps 5 and 8 of the synthesis are shown in Scheme 1-3-5 and Scheme 1-3-8, respectively.

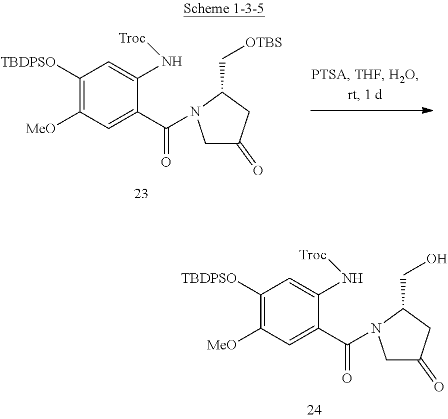

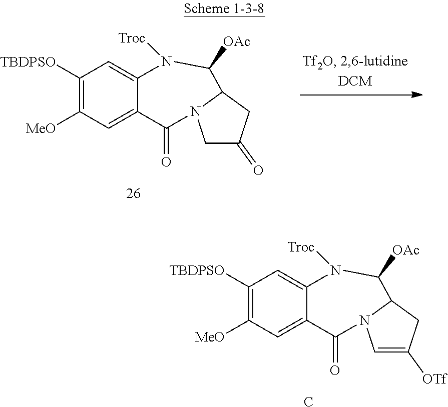

In step 5, as shown in Scheme 1-3-5, compound 23 was reacted with PTSA in THF/water under room temperature for 1 day to form compound 24 (6.60 g in 68% yield). In step 8, compound 26 was mixed with Tf₂O (9.0 eq.) and pyridine (11.0 eq.) under room temperature and stirred for 6 hours. Compound C was successfully synthesized after 6 hours (18 mg in 25% yield).

### Step 4. Synthesis of Compound D and PBD Dimer—NH₂

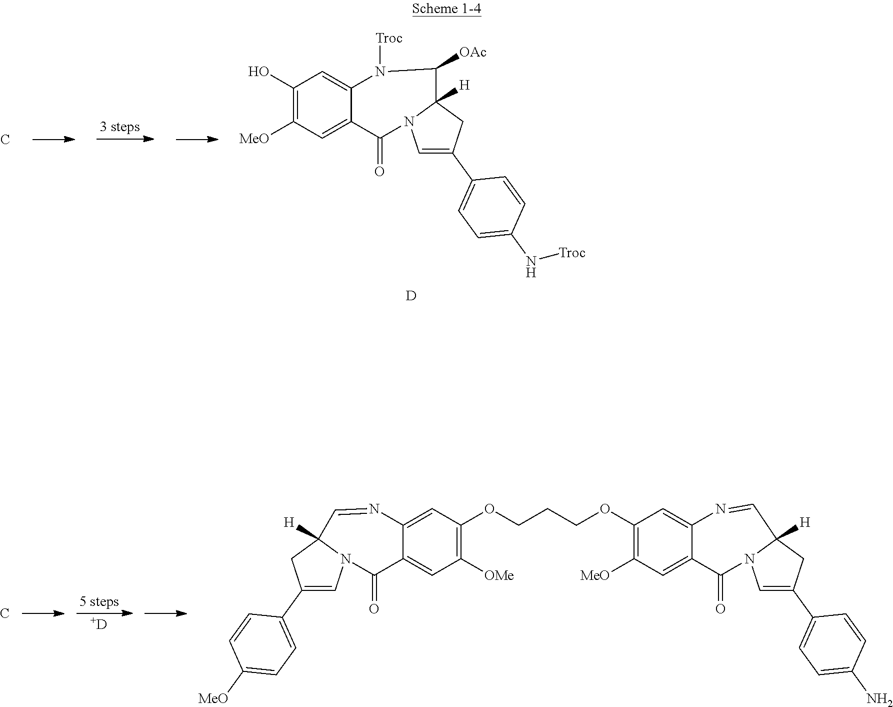

Starting with compound C, compound D was synthesized. By combining compound C and compound D, PBD dimer—NH₂ (compound X-1) was successfully synthesized.

### Example 2. Synthesis of Compound of Formula (A-1)

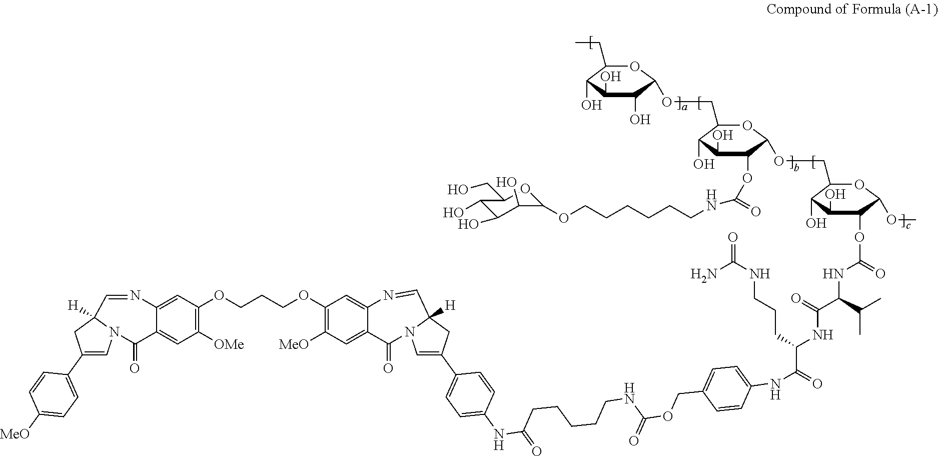

### Step 1. Synthesis of 2,2,2-trichloroethyl (11S,11aS)-11-acetoxy-8-(3-bromopropoxy)-7-methoxy-2-(4-methoxyphenyl)-5-oxo-11,11a-dihydro-1H-benzo[e]pyrrolo[1,2-a][1, 4]diazepine-10(5H)-carboxylate (Compound 40)

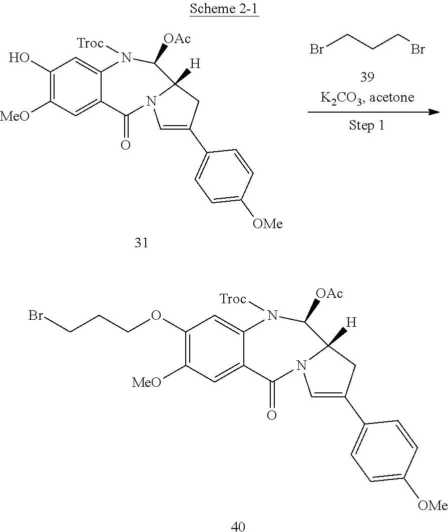

As shown in Scheme 2-1, compound 31 was mixed with 1,3-dibromopropane (compound 39) and K₂CO₃ and the mixture was stirred in acetone. Compound 40 was successfully obtained.

### Step 2. Synthesis of Compound 63

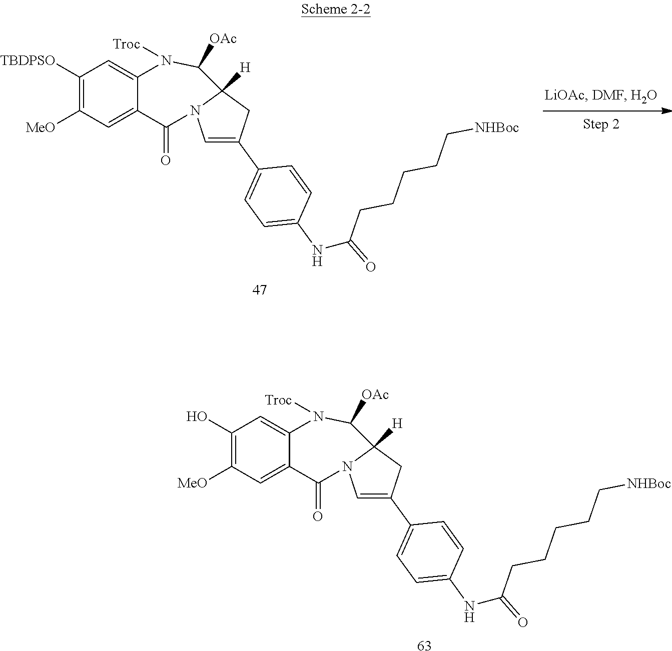

As shown in Scheme 2-2, compound 47 was mixed with LiOAc and the mixture was stirred in DMF/water. Compound 63 was successfully obtained.

### Step 3. Synthesis of Compound 64

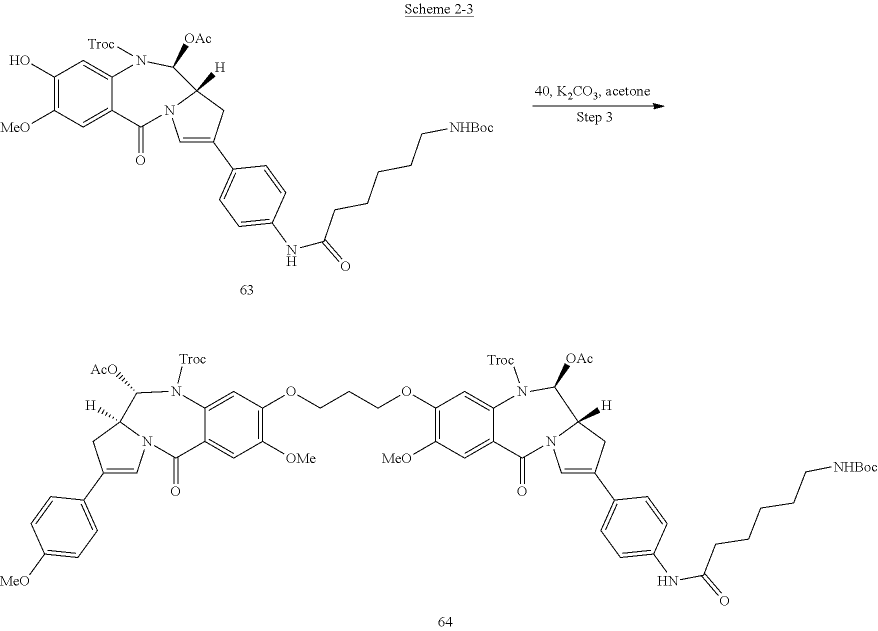

As shown in Scheme 2-3, compound 40 and compound 63 was mixed with K₂CO₃ and was stirred in acetone. Compound 64 was successfully obtained.

### Step 4. Synthesis of Compound 65

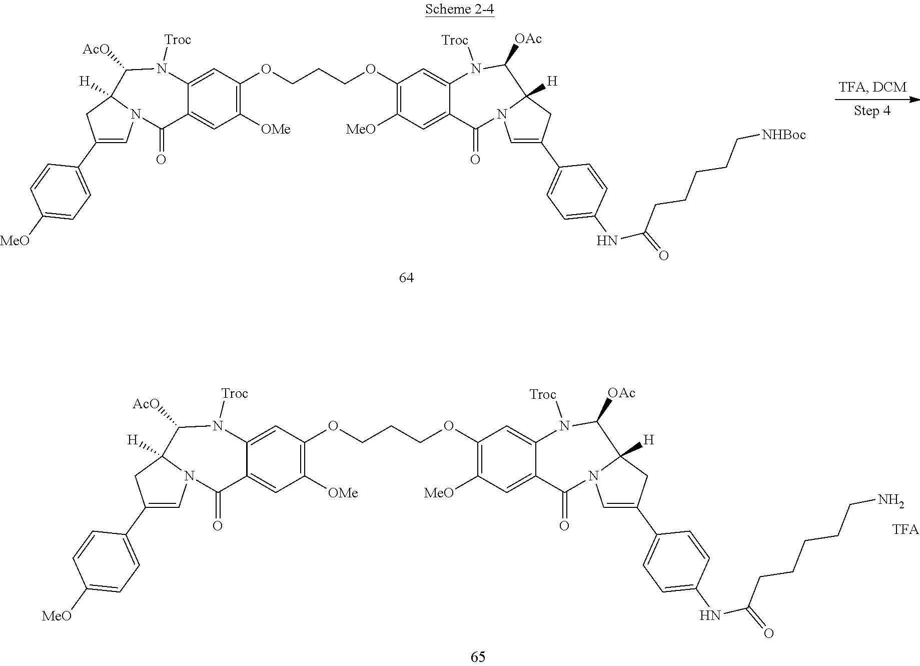

The —NHBoc group in linker moiety was successfully deprotected as shown in Scheme 2-4. Compound 64 was reacted with TFA and DCM, and compound 65 was successfully obtained.

### Step 5. Synthesis of Protected PBD Dimer with Protected Linker (Compound 51)

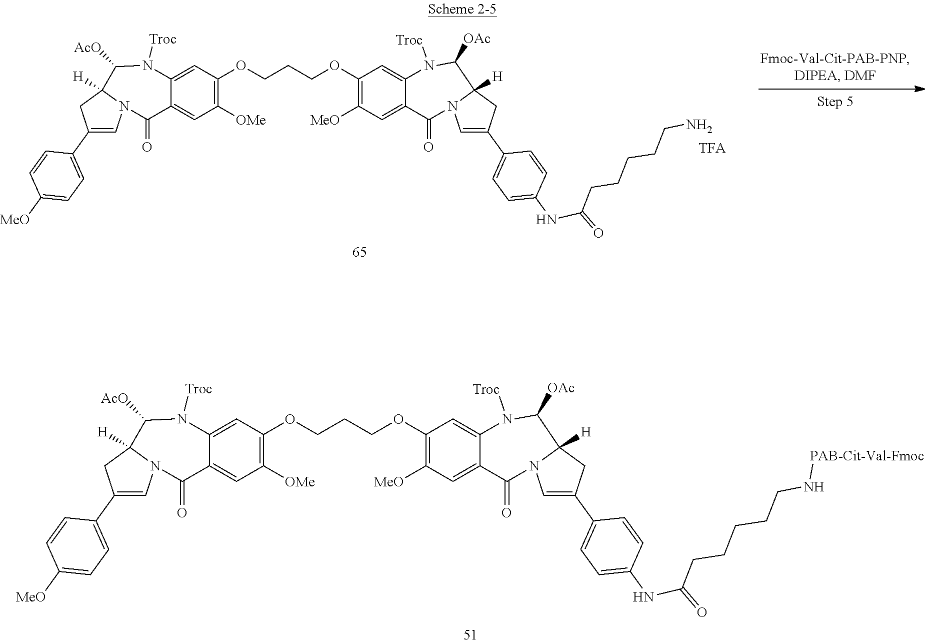

As shown in Scheme 2-5, compound 65 was reacted with Fmoc-Val-Cit-PNP, DIPEA and DMF, and compound 51 was successfully obtained (32 mg in 78% yield).

### Step 6. Synthesis of Protected PBD Dimer with Deprotected Linker (Compound X-2)

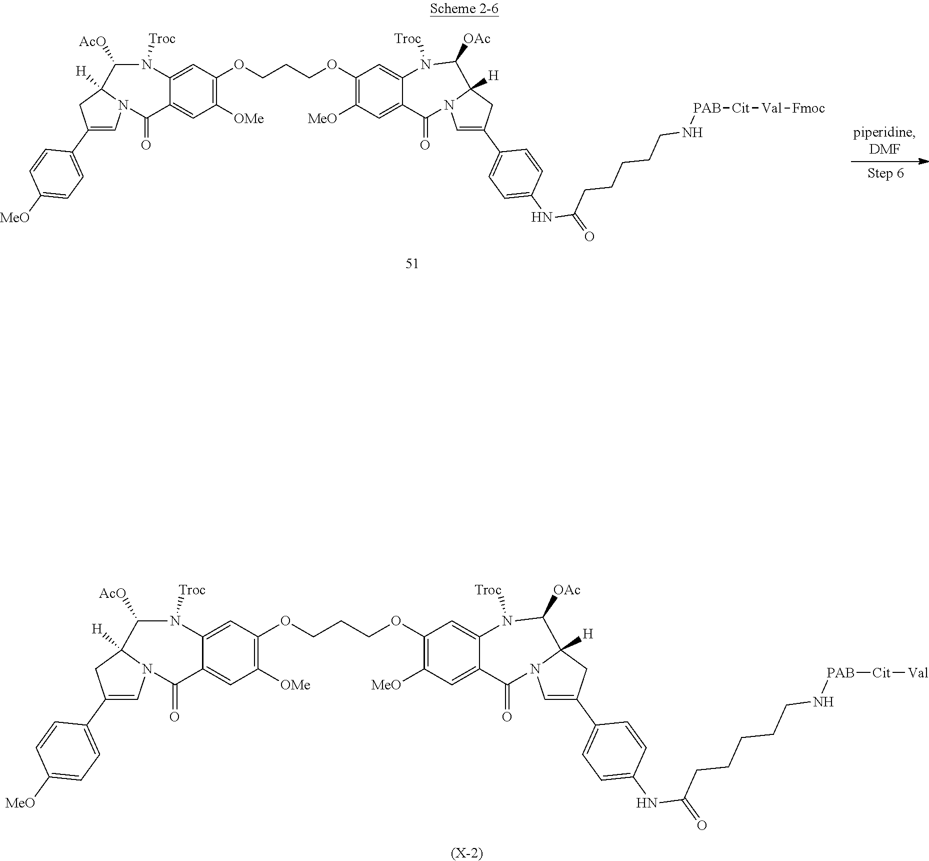

As shown in Scheme 2-6, compound 51 was mixed with piperdine in DMF, and compound (X-2) was successfully obtained (7 mg in 79% yield).

### Step 7. Connecting Compound (X-2) to Backbone

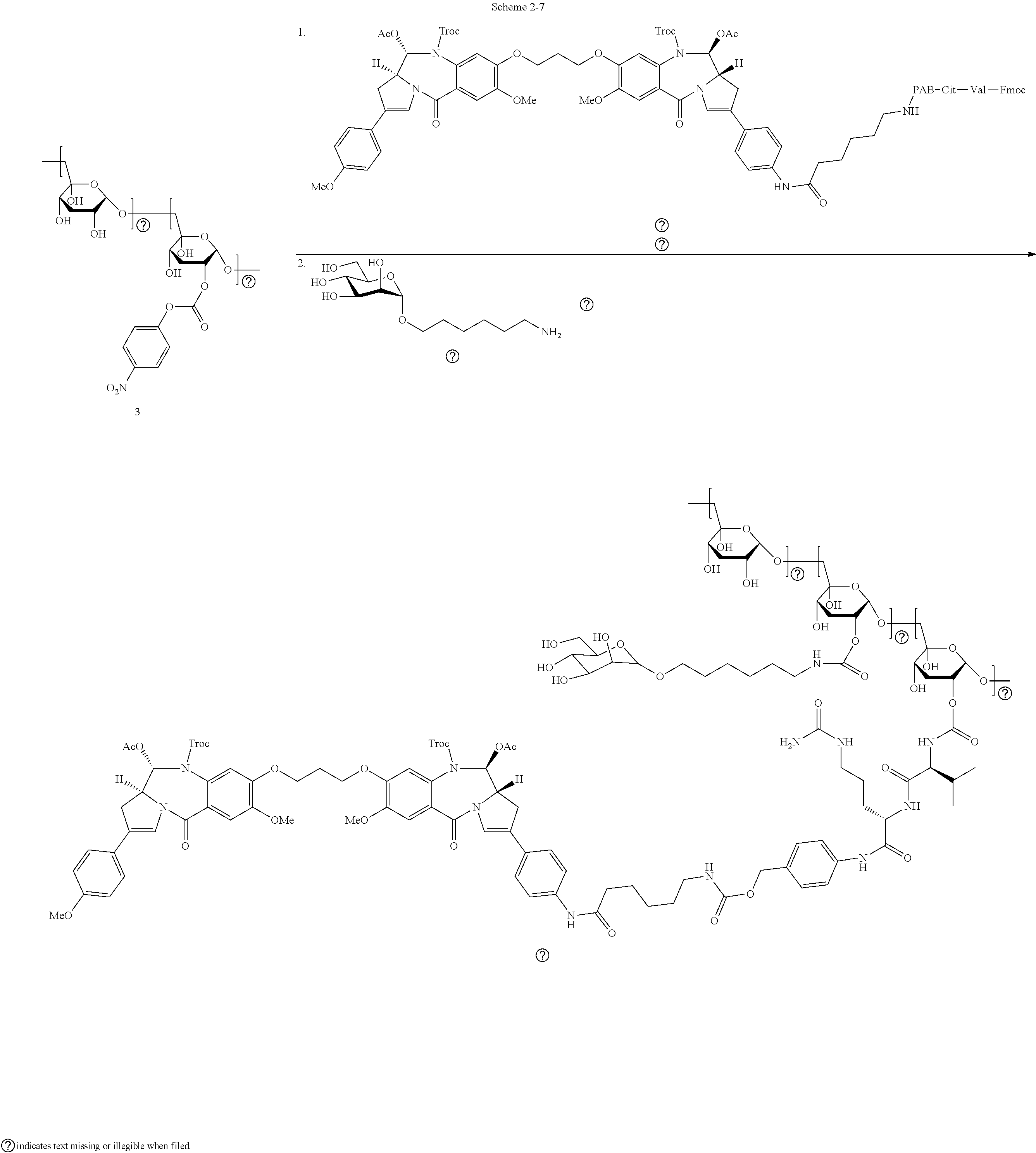

Following Scheme 2-7, the as-synthesized compound (X-2) was first reacted with glucan backbone in DIPEA, HOBt, DMSO, pyridine under room temperature for 90 min. Then the targeting moiety-targeting linker (compound 4) was added to the reaction mixture and the mixture was stirred under room temperature for 20 hours. 15 mg of Compound 69 was successfully obtained. The as-synthesized compound 69 had 3 mol % PBD dimer and 12 mol % mannose.

### Step 8. Synthesis of the Compound of Formula (A-1)

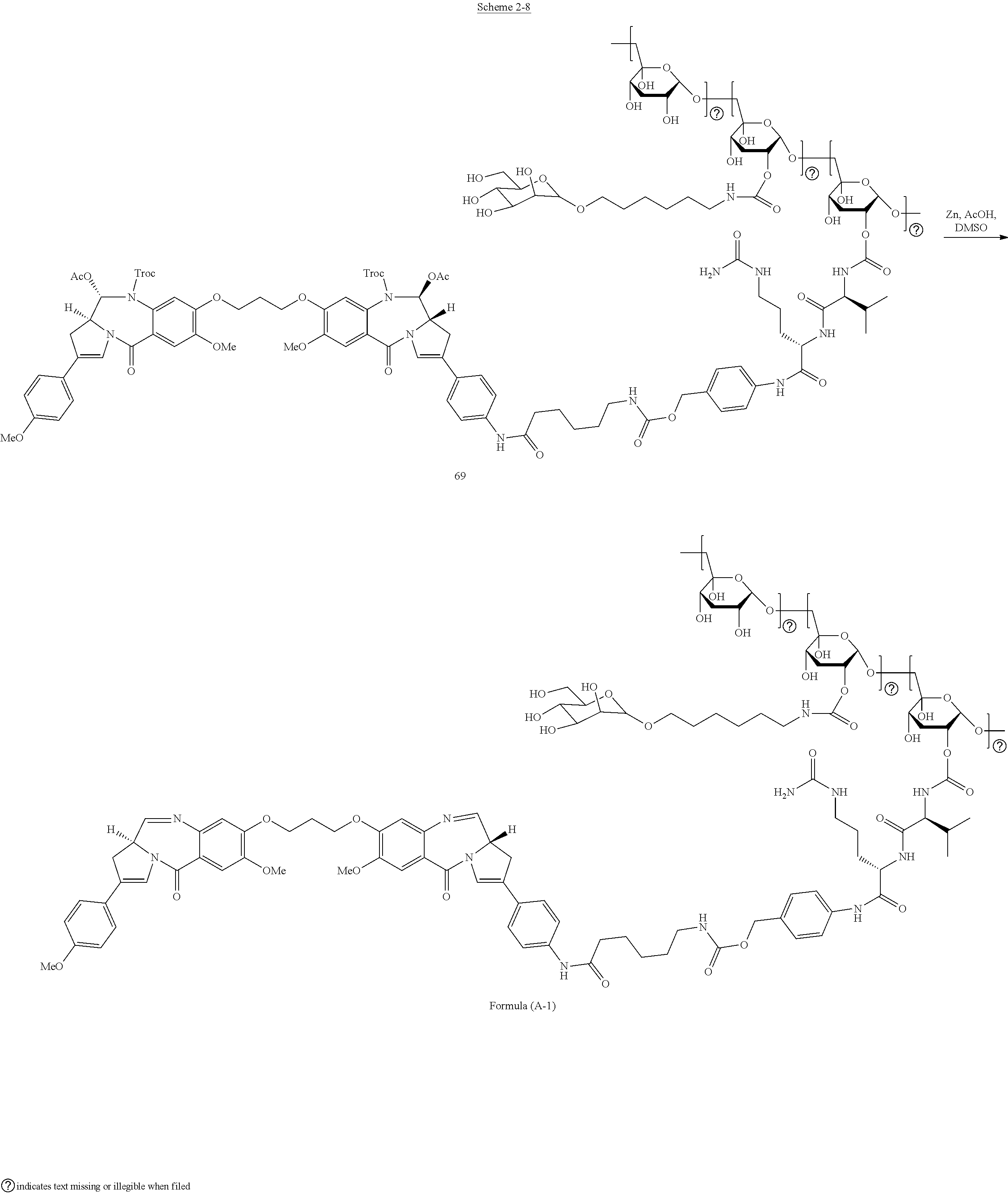

The precursor of the compound of Formula (A-1) (Compound 69) was first synthesized from compound (X-2) based on Scheme A-1. Compound 69 was then reacted with Zn, AcOH, and DMSO to afford the compound of Formula (A-1) (9 mg). The as-synthesized compound of Formula (A-1) had ~3 mol % PBD dimer and ~12 mol % mannose, as determined by NMR.

### Example 3. Synthesis of Compound of Formula (A-2)

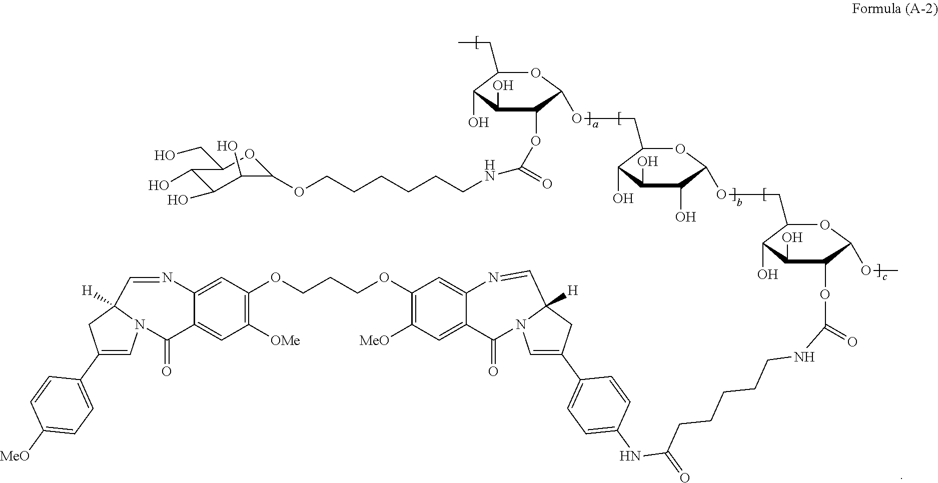

The compound of Formula (A-2) was synthesized with similar procedures as the compound of Formula (A-1) but with a non-cleavable linker. Following Example 2, steps 1-4, compound 65 was obtained, which was a precursor of the active component-payload linker moiety in the compound of Formula (A-2). Compound 65 was then attached onto the backbone as illustrated in Scheme A-1. Following the attachment, the protections groups on the PBD dimer were removed following similar procedures as Example 2, step 8. Compound of Formula (A-2) was obtained.

### Example 4. Selectivity and Toxicity of Compound of Formula (A-1) and Formula (A-2) Towards Mouse Macrophages

The synthesized compound of Formula (A-1) and compound of Formula (A-2) were tested for their selectivity and toxicity towards mouse macrophages. Poly-L-Lysine coat 6-well plate (all wells) was prepared by adding 2 mL of sterile water to each well, further adding 20 μl of poly-L-lysine stock solution (1 mg/ml), mixing, and leaving the wells at 37 °C for 1 hour or overnight. The complete media was prepared by thawing MaGS, FBS, P/S (Sciencell) at 37 °C and when MaGS was completely dissolved, adding them all to Macrophage Medium (MaM). The plate wells were rinsed with sterile water (2×), and 3 ml of complete media was added to each well. Cells were completely thawed in 37 °C water bath and were resuspended, and 150 μL of macrophages were added to each well and dispersed gently by rocking. The cells in wells were left to settle and attach for a minimum of 16 hours, then culture media was changed.

Once cells were attached, for M1 differentiation, LPS (6.25 μl of 500× stock)+50 ng/ml mouse IFN-gamma (1.5 μl of 100 μg/ml stock) were added; and for M2 differentiation, 10 ng/ml IL-4 (0.3 μl of 0.1 mg/ml stock)+10 ng/ml IL-13 (0.3 μl of 0.1 mg/ml stock) were added. 3 Days later cells were detached from dish using 0.05% trypsin for less than 10 min at 37 °C The mixture was collected for staining procedure (spin down resuspend in staining buffer 100 μl). The detachment was achieved by macrophage detachment solution promo cell C-41330 (>1 hour to detach). The recommended CD206 staining is 10 μL/10⁶ cells, and for this experiment, 2 μL was used. The cells are from Science catalog #: M1920-57, and all these cell conditions and differentiation protocols were following the manufacture instructions. Polarization using bone marrow derived. M1 differentiation was achieved by 1×LPS and 50 n/mL mouse IFN-gamma. M2 differentiation was achieved by 10 ng/mL IL-4 and 10 ng/mL IL-13. The differentiation process took three days, and then after another 48 hours, and cells were treated with the compound of Formula (A-1) and compound of Formula (A-2), and 3 μM staurosporine toxicity control, followed by CellTier Flo viability readout (Spectramax).

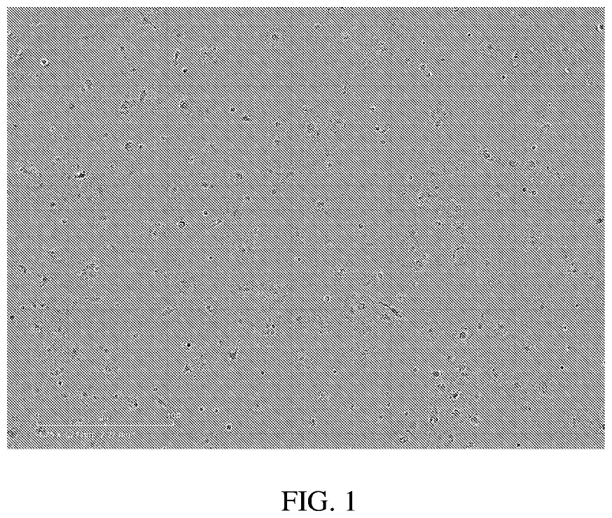

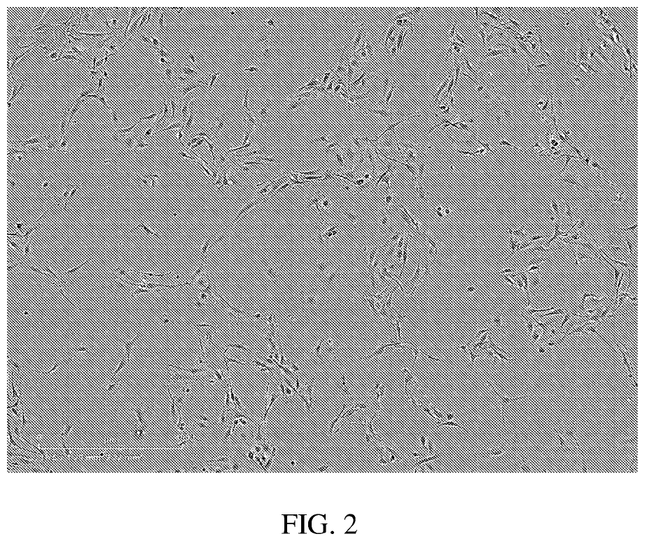

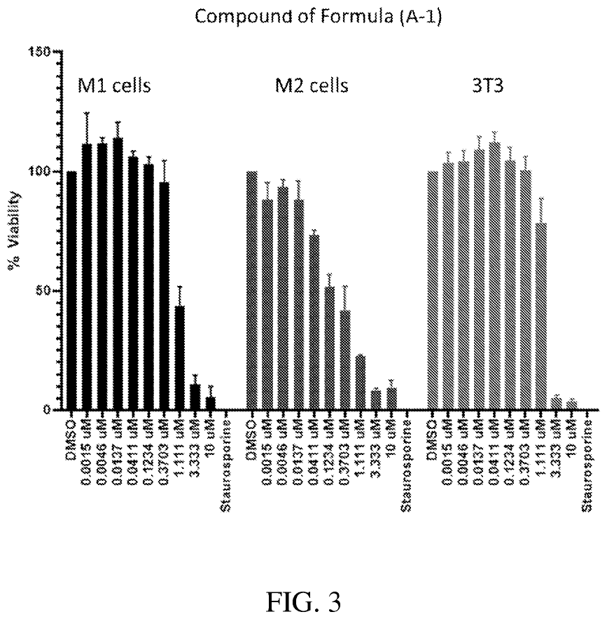

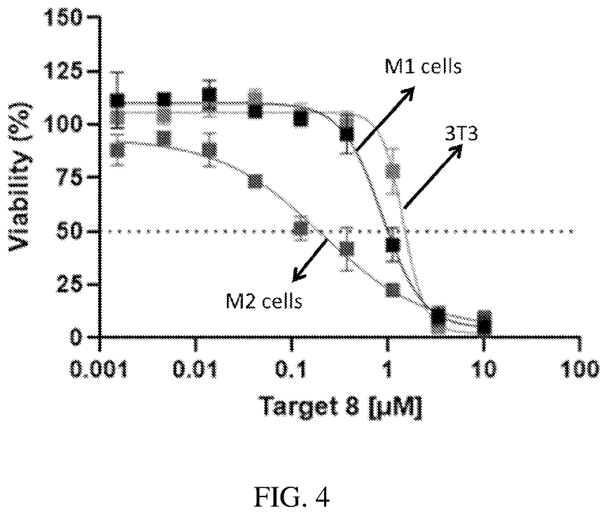

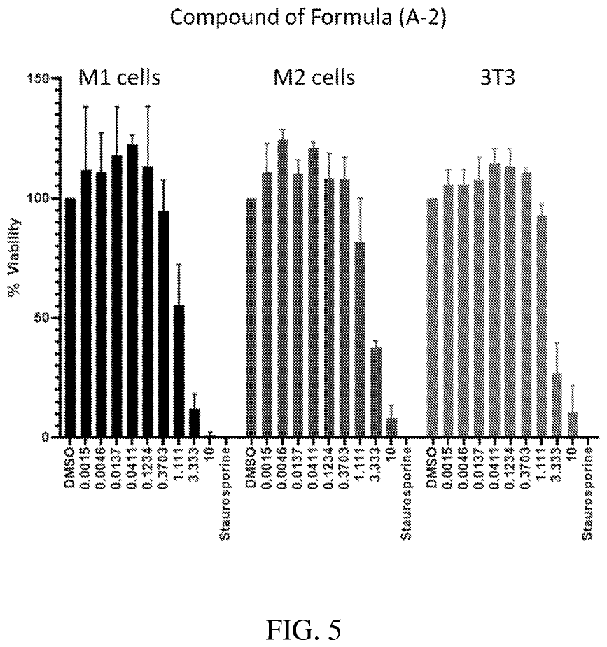

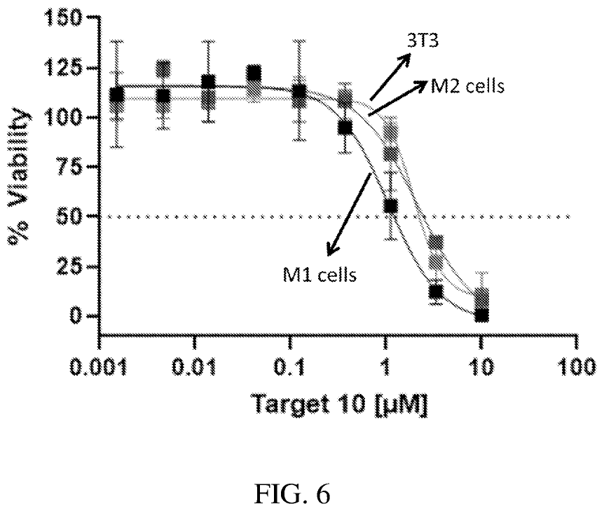

Images of mouse M1 and M2 morphology are shown in FIG. 1 and FIG. 2, respectively. Results of mouse cells treated with the compound of Formula (A-1) are summarized in FIG. 3, FIG. 4 and Table 4-1. Results of mouse cells treated with the compound of Formula (A-2) are summarized in FIG. 5, FIG. 6 and Table 4-2. As seen in Table 4-1, M2 macrophages are 4× more sensitive to the cytotoxicity of A1 than are M1 macrophages, and 7 times more sensitive than 3T3 cells. This is consistent with the fact that M2 macrophages express higher levels of CD206 (the A1 targeted receptor) than do M1 macrophages, and that 3T3 cells do not express the A1 target. This result is consistent with higher uptake by M2 cells at a lower concentration of AL. The IC50 results in Table 4-2 indicate that none of the three cell types are strongly impacted by A2, indicating that a cleavable linker confers greater biological activity for both dividing and non-dividing cells.

TABLE 4-1

| | M1 | M2 | 3T3 |
|---|---|---|---|
| Viability Bottom (%) | 4.614 | 4.007 | 2.388 |
| Viability Top (%) | 110.2 | 93.64 | 105.7 |
| IC₅₀ (μM) | 0.8641 | 0.2020 | 1.439 |
| HillSlope | −2.054 | −0.8116 | −3.857 |

TABLE 4-2

| | M1 | M2 | 3T3 |
|---|---|---|---|
| Viability Bottom (%) | −2.971 | −4.027 | 9.489 |
| Viability Top (%) | 116 | 115.8 | 109.7 |
| IC₅₀ (μM) | 1.055 | 2.131 | 1.946 |
| HillSlope | −2.054 | −0.8116 | −3.857 |

### Example 5. Maximum Tolerated Dose (MTD) of TA in CD-1 Mice Following IV Administration of the Compound of Formula (A-1)

The maximum tolerated dose of Formula (A-1) was examined in CD-1 mice. Twenty five (25) female CD-1 mice were divided into 5 groups and all animals were monitored until 7 days. After 7 days, animals were weighed and assigned to treatment groups based on their body weight using a randomization procedure. The start of the study where randomization and dosing treatment begins was designated as Day 0. Mice were administered the compound of Formula (A-1) at dose of 0.3 mg/kg (G1); 1.0 mg/kg (G2); 3.0 mg/kg (G3); 10.0 mg/kg (G4); or 30.0 mg/kg (G5) in a single slow IV bolus. Animals were monitored closely after dosing. Weight and food consumption was monitored and recorded for all mice for seven days. At Day 7, mice were sacrificed and a necropsy was performed. Serum for clinical chemistry and whole blood for hematology were collected from all mice.

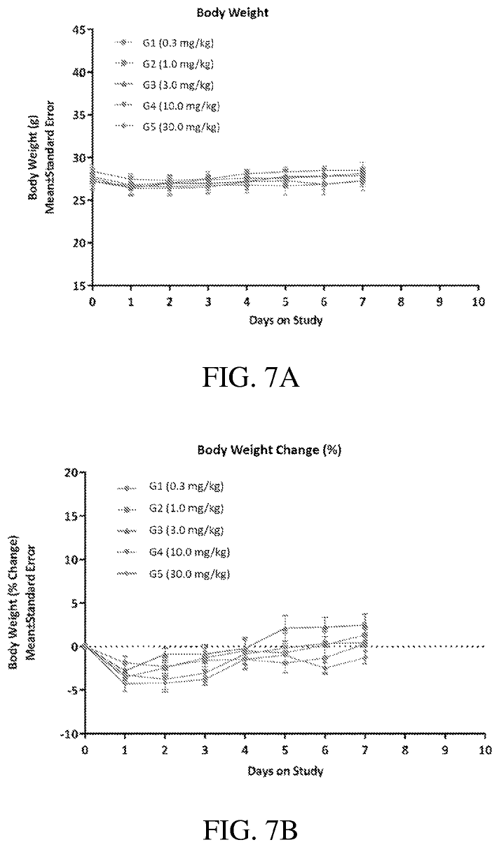

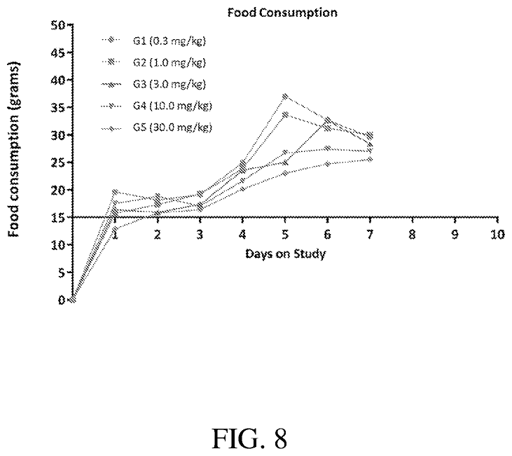

FIGS. 7A and 7B illustrate the change in body weight and percent of body weight change across all five treatment groups. FIGS. 7A and B show that the animals experienced less than a 5% decrease in bodyweight across all groups, and regained the weight gradually over 7 days. Similarly, FIG. 8 shows little difference in food consumption between the five treatment groups. All organs were observed to be normal during the gross necropsy performed at Day 7.

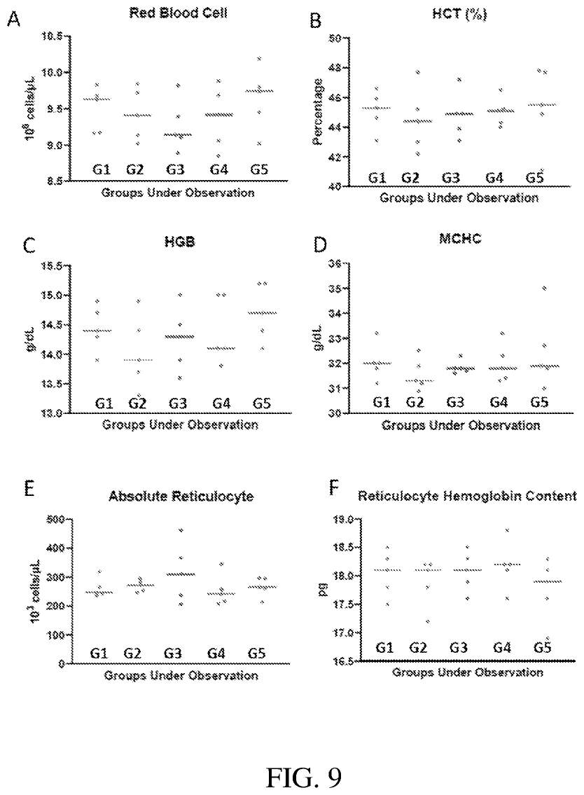

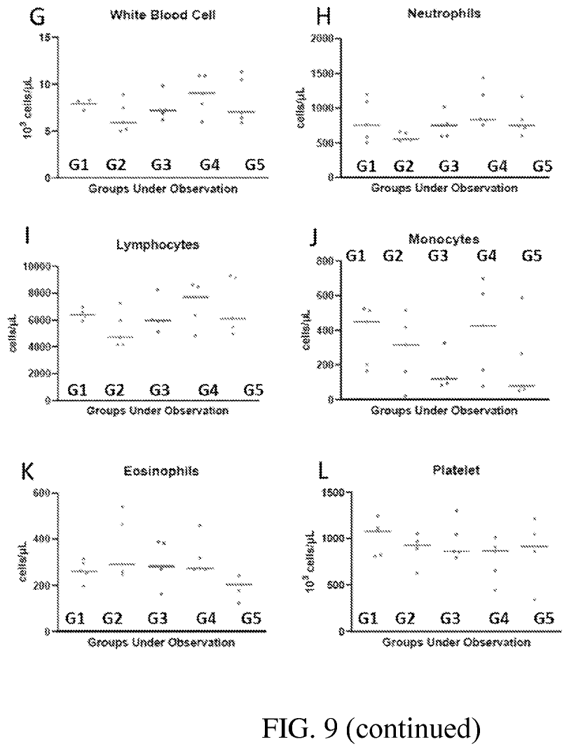

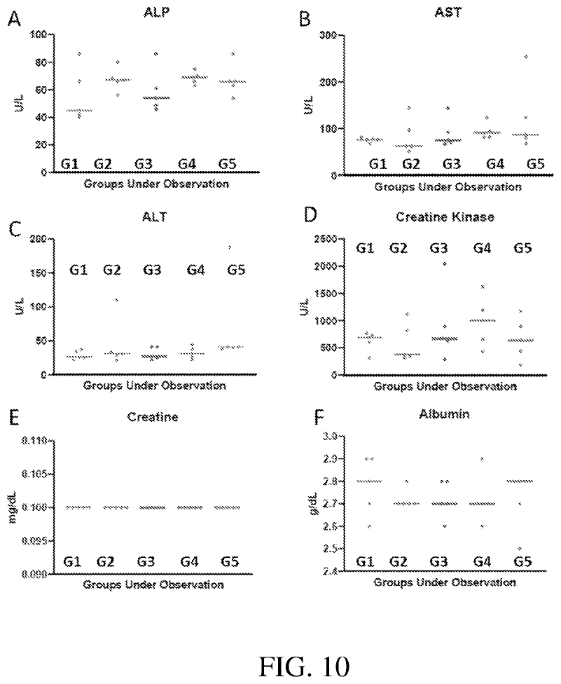

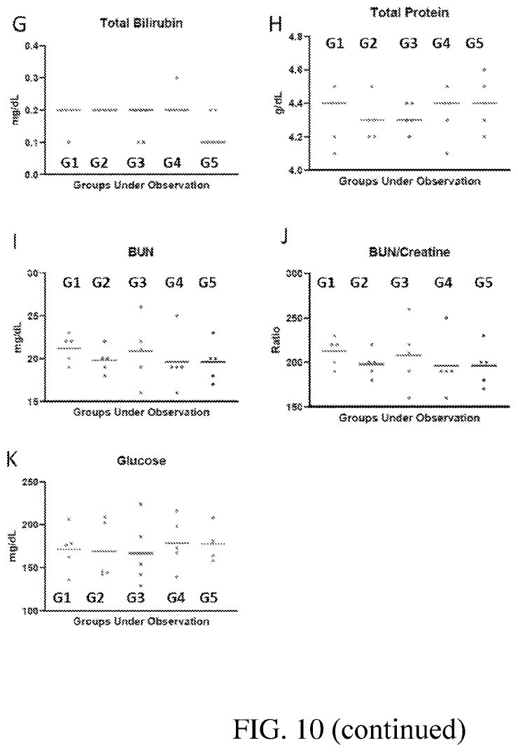

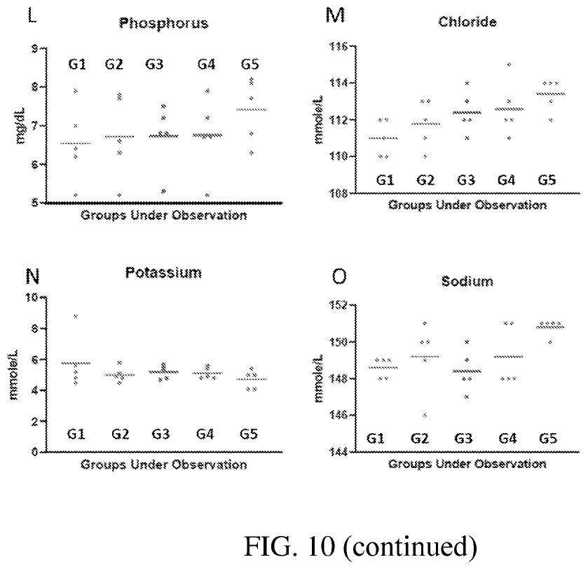

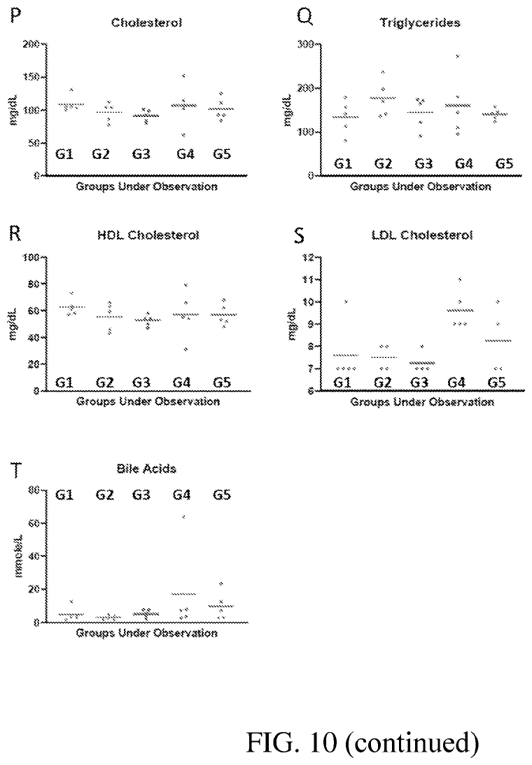

The analysis results of blood hematology and serum clinical chemistry did not show any significant difference between all groups. FIG. 9A-9L show that none of the treatment groups exhibit any differences hematology parameters. Similarly, FIG. 10A-10T show no differences in serum components between the treatment groups.

These data indicate that the compound of Formula (A-1) was tolerated over a range from 0.3 mg/kg to 30 mg/kg. The maximum tolerated dose of Formula (A-1) in CD-1 mice was not defined in this study. It is anticipated that mice could be treated at least up to once per week at 30 mg/kg in a mouse tumor model.

### Example 6. Anti-Cancer Efficacy of the Compound of Formula (A-1) In Vivo

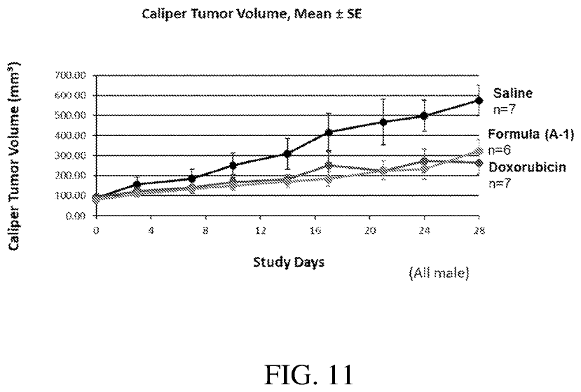

The initial anti-cancer efficacy of compound of Formula (A-1) was examined in a PDX myxofibrosarcoma model. Twenty (20) male PDX mice were randomly divided according to tumor size into three different treatment groups and were administered either saline, doxorubicin, or Formula (A-1). The mice were administered the respective treatment intravenously, once a week at a concentration of 1 mg/kg for three weeks for a total of three doses (Days 0, 7, and 14). As seen from FIG. 11, the tumor volume of mice administered Formula (A-1) was comparable with the tumor volume of mice administered doxorubicin. Both the Formula (A-1)-treated and doxorubicin-treated mice experienced lower tumor burden than mice treated with saline.

1. A composition comprising:
i) a tumor-associated macrophage-targeting moiety;
ii) a glucan backbone, wherein the glucan backbone comprises a plurality of backbone monomers;
iii) an active component comprising a pyrrolobenzodiazepine (PBD) dimer, wherein the active component is coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety.

2. The composition of claim 1, wherein the PBD dimer is a compound of Formula (I): 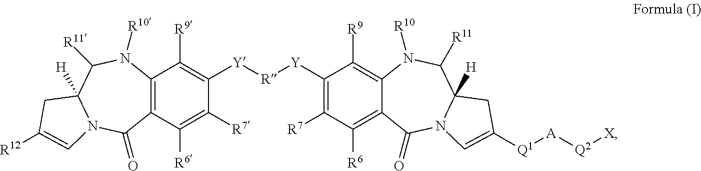
wherein:
A is a C₆-C₁₀ arylene group and X is selected from the group consisting of 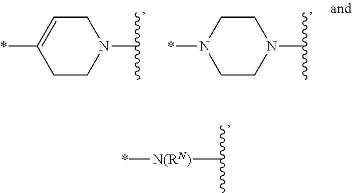
wherein R^{N} is selected from the group consisting of H and C₁-C₄ alkyl; and the asterisk indicates the point of attachment to Q²
Q¹ and Q² are each independently selected from a single bond, —Z—(CH₂)ₙ—, or —CH═CH—, wherein Z is selected form the group consisting of a single bond, O, S, and NH, and subscript n is from 1 to 3;
R¹² is a C₆-C₁₀ aryl group, optionally substituted by one or more substituents selected form the group consisting of —OR^{12a} and —COOR^{12b}, wherein R^{12a} and R^{12b} are each independently H or C₁-C₆ alkyl;
R⁶, R⁷, and R⁹ are each independently selected from the group consisting of H, R^{b}, —OR^{b}, —SR^{b}, —NR^{b}R^{c}, —NO₂—, —SnMe₃, and halo;
wherein R^{b} and R^{c} are each independently selected from the group consisting of C₁-C₆ alkyl, 4- to 10-membered heterocyclyl, and C₅-C₂₀ aryl, each of which is optionally substituted;
R¹⁰ and R¹¹ are each independently selected from the group consisting of H, —OR^{11a}, and —SO_{Z}M, wherein R^{11a} is H or C₁-C₆ alkyl, subscript Z is 2 or 3, and M is a pharmaceutically acceptable cation; or
R¹⁰ and R¹¹ form a nitrogen-carbon double bond between the nitrogen and carbon atoms to which they are attached;
R¹¹ is a C₃-C₁₂ alkylene, the chain of which is optionally interrupted by one or more heteroatoms selected from the group consisting of 0, S, and NR^{N2} wherein R^{N2} is H or C₁-C₄ alkyl, and/or by an C₅-C₁₀ arylene;
Y and Y′ are each independently selected from the group consisting of O, S, and NH; and
R^{6′}, R^{7′}, R^{9′}, R^{10′}, and R^{11′} are selected from the same groups as R⁶, R⁷, and R⁹, R¹⁰, and R¹¹, respectively.

3. The composition of claim 1 or 2, wherein the plurality of backbone monomers comprises a plurality of D-glucose monomers in a α-1,6 glycosidic linkage or beta-1,4 glycosidic linkage.

4. The composition of claim 3, wherein the plurality of D-glucose monomers is n, wherein n=16 to 111.

5. The composition of claim 3 or 4, wherein the plurality of D-glucose monomers is n, wherein n=50 to 65.

6. The composition of any one of claims 1 to 5, wherein the glucan backbone is a linear dextran molecule.

7. The composition of any one of claims 1 to 5, wherein the glucan backbone is a cyclodextrin molecule, wherein n=6 to 16.

8. The composition of any one of claims 1 to 7, wherein the tumor-associated macrophage-targeting moiety comprises mannose, galactose, collagen, fucose, sulfated N-acetylgalactosamine, N-acetylglucosamine, luteinizing hormone, thyroid stimulating hormone, phospholipase A2 or fragments thereof, or a chondroitin sulfate.

9. The composition of any one of claims 1 to 8, wherein the targeting moiety is a mannose.

10. The composition of claim 9, wherein the ratio of mannose to backbone monomers is about 1 to 5 to about 1 to 25.

11. The composition of claim 9 or 10, wherein the ratio of mannose to backbone monomers is about 1 to 6 to about 1 to 19.

12. The composition of any one of claims 1 to 8, wherein the degree of substitution of mannose on a cyclodextrin ranges from about 0.1 to about 7.

13. The composition of any one of claims 1 to 9 or 12, wherein the degree of substitution of mannose on a cyclodextrin ranges from about 0.5 to 5.

14. The composition of any one of claims 1 to 13, wherein the targeting linker is connected to the glucan backbone through the oxygen atom of the carbamate group.

15. The composition of any one of claims 1 to 14, wherein the chain moiety of the targeting linker comprises a C₃-C₇ alkylene chain.

16. The composition of any one of claims 1 to 15, wherein the chain moiety of the targeting linker comprises a C₆-alkylene moiety.

17. The composition of any one of claims 1 to 16, wherein the chain moiety of the targeting linker is an unsubstituted C₆-alkylene moiety.

18. The composition of any one of claims 1 to 17, wherein the carbon atom of the carbamate group of the targeting linker is the only sp2-hybridized carbon when said linker is attached to mannose.

19. The composition of any one of claims 1 to 18, wherein the tumor-associated macrophage-targeting moiety is a moiety targeting CD205 (DEC205), CD206, CD207 (langerin), CD209 (DC-SIGN), CD280 (ENDO180), or CD301 (MGL).

20. The compound of claim 19, wherein the tumor-associated macrophage-targeting moiety is a CD206 targeting moiety.

21. The composition of any one of claims 1 to 20, wherein the composition has a molar ratio molar ratio between the TAM-targeting moiety and the PBD toxin from about 1:1 to about 1:10.

22. The composition of any one of claims 1 to 20, wherein the molar ratio between the TAM-targeting moiety and the PBD toxin is about 1:1, about 1:2, or about 1:3.

23. The composition of claim 1, wherein the active component is coupled to the glucan backbone via a payload linker.

24. The composition of claim 23, wherein the payload linker is a non-cleavable linker.

25. The composition of claim 24, wherein the payload linker comprises a carbamate group and a chain moiety, wherein the carbamate group is connected to the backbone monomer and the chain moiety connects the carbamate group and the active component.

26. The composition of claim 23, wherein the payload linker is a cleavable linker.

27. The composition of claim 26, wherein the cleavable linker is capable of being cleaved by a protease.

28. The composition of claim 27, wherein the protease is a lysosomal protease or an endosomal protease.

29. The composition of claim 26, wherein the cleavable linker is capable of being cleaved by a pH change.

30. The composition of claim 26, wherein the payload linker comprises a Val-Cit moiety.

31. A compound of formula (A-1):

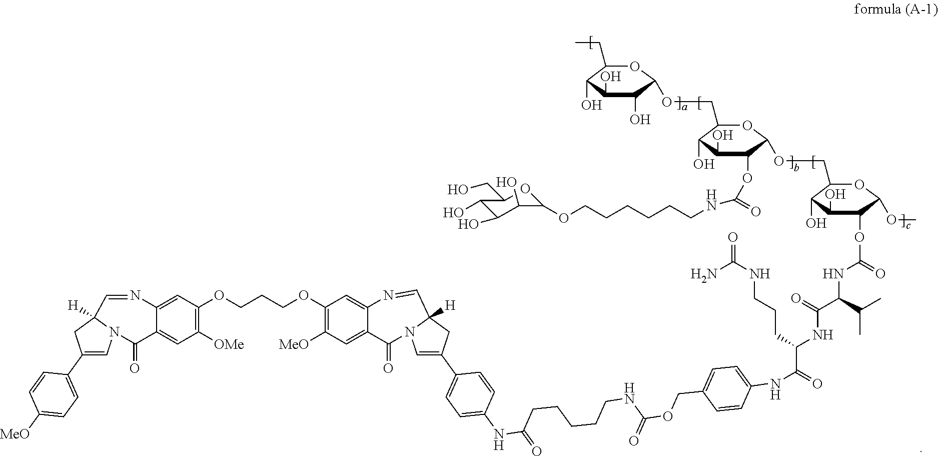

32. A compound of formula (A-2):

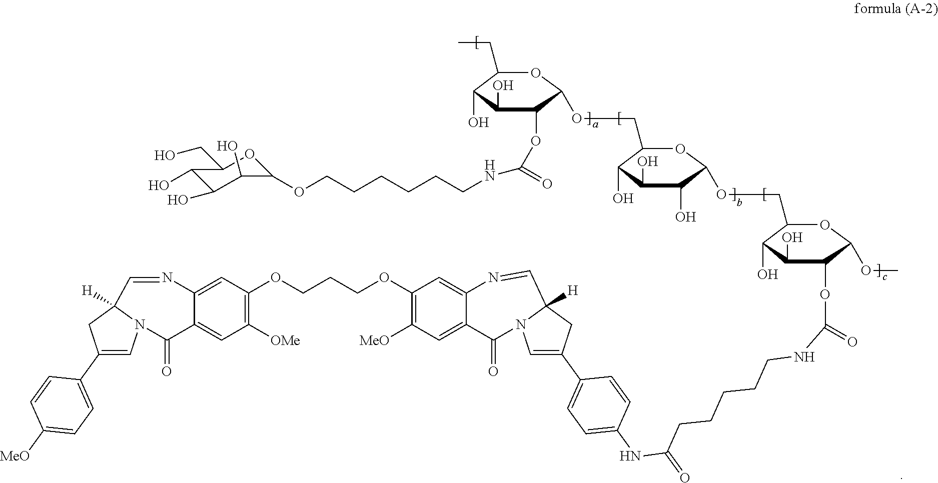

33. A method of delivering an agent to a tumor-associated macrophage comprising contacting said macrophage with the composition of any one of claims 1 to 30 or the compound of claim 31 or 32.

34. A method of treating cancer in a subject in need thereof, comprising administering to the subject a therapeutically effective amount of the composition of any one of claims 1 to 30 or the compound of claim 31 or 32.

35. The method of claim 34, wherein the cancer is selected from the group consisting of: carcinoma, lymphoma, blastoma, sarcoma, leukemia, lymphoid malignancies, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

36. The method of claim 34 or 35, wherein the cancer is sarcoma or glioblastoma.

37. The method of any one of claims 34 to 36, wherein the cancer is soft tissue sarcoma.

38. A method of treating non-malignant tumor in a subject in need thereof, comprising administering to the subject a therapeutically effective amount of the composition of any one of claims 1 to 30 or the compound of claim 31 or 32.

39. The method of claim 38, wherein the non-malignant tumor is meningiomas hemangioblastoma or giant cell tumor.

40. The method of any one of claims 34 to 37, wherein the subject is administered a second therapeutic agent, an adjuvant therapy, or radiation therapy.
